(19) United States
(12) Patent Application Publication
(10) Pub. No.: US 2013/0072713 A1
(43) Pub. Date: Mar. 21, 2013

(54) METHOD FOR PRODUCING METHIONINE

(71) Applicants: Koji HAGIYA, Osaka (JP); Kazuyasu TANI, Osaka (JP)

(72) Inventors: Koji HAGIYA, Osaka (JP); Kazuyasu TANI, Osaka (JP)

(21) Appl. No.: 13/616,569

(22) Filed: Sep. 14, 2012

(30) Foreign Application Priority Data
Sep. 16, 2011 (JP) 2011-202871

(51) Int. Cl. C07C 319/12

(52) U.S. Cl. 562/559

(57) ABSTRACT

A novel method for producing methionine without using hydrogen cyanide as a raw material has been demanded. A method for producing methionine including a step A of oxidizing 4-methylthio-2-oxo-1-butanal in the presence of an alcohol; a step B of hydrolyzing a 4-methylthio-2-oxo-butanoic acid ester obtained in the step A; and a step C of subjecting 4-methylthio-2-oxo-butanoic acid obtained in the step B to reductive amination.

### BACKGROUND OF THE INVENTION

The present invention relates to a method for producing methionine.

### DESCRIPTION OF THE RELATED ART

Methionine is an essential amino acid and an important compound to be used also as a feed additive.

As methods for producing methionine, for example, Industrial Organic Chemistry, Tokyo Kagaku Dojin, p. 273-275 (1978) discloses a method of causing a reaction of 3-methylthiopropionaldehyde obtained by adding methanethiol to acrolein and hydrogen cyanide to obtain 2-hydroxy-4-methylthiobutyronitrile, which is reacted with ammonium carbonate to produce a substituted hydantoin, which is then hydrolyzed with an alkali.

### SUMMARY OF THE INVENTION

The method disclosed in Industrial Organic Chemistry, Tokyo Kagaku Dojin, p. 273-275 (1978) uses hydrogen cyanide, as a raw material, which requires to be handled carefully, and when hydrogen cyanide is handled, sufficiently attentive management and equipment for the management are required.

Under the above-mentioned situation, a novel method for producing methionine without using hydrogen cyanide as a raw material has been demanded.

Inventors of the present invention have made various investigations to solve the problems and consequently have completed the present invention.

That is, the present invention is as follows.

[1] A method for producing methionine including

a step A of oxidizing 4-methylthio-2-oxo-1-butanal in the presence of an alcohol;

a step B of hydrolyzing a 4-methylthio-2-oxo-butanoic acid ester obtained in the step A; and

a step C of subjecting 4-methylthio-2-oxo-butanoic acid obtained in the step B to reductive amination.

[2] The method according to [1], wherein the step A is carried out by causing a reaction of 4-methylthio-2-oxo-1-butanal, an alcohol, and an oxidizing agent in the presence of a carbene catalyst.
[3] The method according to [2], wherein the carbene catalyst in the step A is at least one selected from the group consisting of:
a compound obtained by a reaction of a compound represented by a formula 2-1)

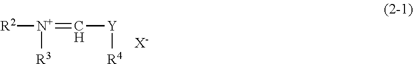

wherein R² represents an optionally substituted alkyl group or an optionally substituted aryl group; R³ and R⁴ each independently represent an optionally substituted alkyl group or an optionally substituted aryl group or R³ and R⁴ may be bonded together to form an optionally substituted divalent hydrocarbon group or an optionally substituted group represented by —CH═N—; Y represents a group represented by —S— or a group represented by —N(R⁵)—; R⁵ represents an optionally substituted alkyl group or an optionally substituted aryl group or R⁵ may be bonded together with R⁴ to form an optionally substituted divalent hydrocarbon group; and X⁻ represents an anion and a base;
a compound represented by a formula (2-2)

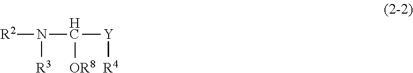

wherein R², R³, R⁴, and Y are the same as described above, respectively; and R⁸ represents an alkyl group;
a compound obtained by decomposing the compound represented by the formula (2-2);
a compound represented by a formula (2-3)

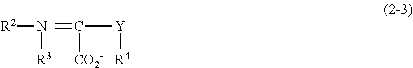

wherein, R², R³, R⁴, and Y are the same as described above, respectively); and
a compound obtained by decomposing the compound represented by the formula (2-3).
[4] The method according to [2] or [3], wherein the oxidizing agent in the step A is at least one selected from the group consisting of oxygen and carbon dioxide.
[5] The method according to any one of [1] to [4], wherein the alcohol is methanol or ethanol.
[6] The method according to any one of [1] to [5], wherein the step C is carried out in the presence of a solvent.
[7] The method according to [6], wherein the solvent in the step C is at least one selected from the group consisting of methanol and water.
[8] The method according to any one of [1] to [7], wherein the step C is carried out by causing a reaction of 4-methylthio-2-oxo-butanoic acid, ammonia, and a reducing agent in the presence of a transition metal.
[9] The method according to [8], wherein the transition metal in the step C is at least one selected from the group consisting of ruthenium, rhodium, palladium, platinum, iridium, nickel, cobalt, and copper.

According to the present invention, a novel method for producing methionine without using hydrogen cyanide as a raw material can be provided.

### DETAILED DESCRIPTION OF THE PREFERRED EMBODIMENTS

Hereinafter, the present invention will be described in detail.

The method for producing methionine of the present invention has a feature of including a step A of oxidizing 4-methylthio-2-oxo-1-butanal in the presence of an alcohol; a step B of hydrolyzing a 4-methylthio-2-oxo-butanoic acid ester obtained in the step A; and a step C of subjecting 4-methylthio-2-oxo-butanoic acid obtained in the step B to reductive amination. Methionine can be produced by carrying out the step A, step B, and step C without using hydrogen cyanide as a raw material.

First, the step A of oxidizing 4-methylthio-2-oxo-1-butanal in the presence of an alcohol will be described. Execution of the step A gives a 4-methylthio-2-oxo-butanoic acid ester.

The step A is carried out by oxidizing 4-methylthio-2-oxo-1-butanal in the presence of an alcohol, and preferably is carried out by oxidizing 4-methylthio-2-oxo-1-butanal, an alcohol, and an oxidizing agent in the presence of a carbene catalyst. Hereinafter, the reaction in the step A may be referred to as an oxidation reaction in some cases.

Examples of the carbene catalyst to be used in the step A include at least one selected from the group consisting of a compound obtained by a reaction of a compound represented by a formula (2-1) (hereinafter, may be referred to as “compound (2-1)” in some cases) and a base; a compound represented by a formula (2-2) (hereinafter, may be referred to as “compound (2-2)” in some cases); a compound obtained by decomposing the compound represented by the formula (2-2); a compound represented by a formula (2-3) (hereinafter, may be referred to as “compound (2-3)” in some cases); and a compound obtained by decomposing the compound represented by the formula (2-3).

In the formula (2-1), examples of the alkyl group in the optionally substituted alkyl group represented by R³ and the optionally substituted alkyl group represented by R⁴ include linear or branched C₁ to C₁₂ alkyl groups such as a methyl group, an ethyl group, a propyl group, an isopropyl group, a butyl group, an isobutyl group, a sec-butyl group, a tert-butyl group, a pentyl group, and a decyl group; and C₃ to C₁₂ cycloalkyl groups such as a cyclopropyl group, a 2,2-dimethylcyclopropyl group, a cyclopentyl group, a cyclohexyl group, and a menthyl group.

Examples of the optional substituent of the alkyl group in R³ and R⁴ include groups selected from the following group G3:

### <Group G3>

a C₆ to C₁₀ aryl group which may have a C₁ to C₁₀ alkoxy group;

a C₁ to C₁₀ alkoxy group which may have a fluorine atom;

a benzyloxy group which may have at least one group selected from the group consisting of a C₁ to C₁₀ alkoxy group, a C₁ to C₁₀ alkyl group, and a C₆ to C₁₀ aryloxy group;

a C₆ to C₁₀ aryloxy group which may have a C₁ to C₁₀ alkoxy group;

a C₆ to C₁₀ aryloxy group which may have a C₆ to C₁₀ aryloxy group;

a C₂ to C₁₀ acyl group which may have a C₁ to C₁₀ alkoxy group;

a carboxyl group; and

a fluorine atom.

In the group GO, examples of the C₆ to C₁₀ aryl group which may have a C₁ to C₁₀ alkoxy group include a phenyl group, a naphthyl group, a 4-methylphenyl group, and a 4-methoxyphenyl group;

examples of the C₁ to C₁₀ alkoxy group which may have a fluorine atom include a methoxy group, an ethoxy group, a propoxy group, an isopropoxy group, a butoxy group, an isobutoxy group, a sec-butoxy group, a tert-butoxy group, and a trifluoromethoxy group;

examples of the benzyloxy group which may have at least one group selected from the group consisting of a C₁ to C₁₀ alkoxy group, a C₁ to C₁₀ alkyl group, and a C₆ to C₁₀ aryloxy group include a benzyloxy group, a 4-methylbenzyloxy group, a 4-methoxybenzyloxy group, and a 3-phenoxybenzyloxy group;

examples of the C₆ to C₁₀ aryloxy group which may have a C₁ to C₁₀ alkoxy group include a phenoxy group, a 2-methylphenoxy group, a 4-methylphenoxy group, and a 4-methoxyphenoxy group;

examples of the C₆ to C₁₀ aryloxy group which may have a C₆ to C₁₀ aryloxy group include a 3-phenoxyphenoxy group; and

examples of the C₂ to C₁₀ acyl group which may have a C₁ to C₁₀ alkoxy group include an acetyl group, a propicnyl group, a benzylcarbonyl group, a 4-methylbenzylcarbonyl group, a 4-methoxybenzylcarbonyl group, a benzoyl group, a 2-methylbenzoyl group, a 4-methylbenzoyl group, and a 4-methoxybenzoyl group.

Examples of the alkyl group having a group selected from the group G3 include a fluoromethyl group, a trifluoromethyl group, a methoxymethyl group, an ethoxymethyl group, a methoxyethyl group, a benzyl group, a 4-fluorobenzyl group, a 4-methylbenzyl group, a phenoxymethyl group, a 2-oxopropyl group, a 2-oxobutyl group, a phenacyl group, and a 2-carboxyethyl group.

In the formula (2-1), examples of the aryl group in the optionally substituted aryl group represented by R³ and the optionally substituted aryl group represented by R⁴ include C₆ to C₁₀ aryl groups such as a phenyl group, a 2-methylphenyl group, a 4-methylphenyl group, and a naphthyl group.

Examples of the optional substituent of the aryl group include groups selected from the above group G3.

Examples of the aryl group having a group selected from the group G3 include a 4-chlorophenyl group and a 4-methoxyphenyl group.

In the formula (2-1), examples of the optionally substituted divalent hydrocarbon group formed by bonding R³ and R⁴ together include an ethylene group, a vinylene group, a propane-1,2-diyl group, a propene-1,2-diyl group, a butane-1,2-diyl group, a 2-butene-1,2-diyl group, a cyclopentane-1,2-diyl group, a cyclohexane-1,2-diyl group, an o-phenylene group, a 1,2-diphenylethylene group, and a 1,2-diphenylvinylene group. Examples of the optional substituent of the divalent hydrocarbon group include groups selected from the above-mentioned group G3. In the formula (2-1), examples of the optional substituent of the group represented by —CH═N— and formed by bonding R³ and R⁴ together include an alkyl group which may have a group selected from the above-mentioned group G3 and an aryl group which may have a group selected from the above-mentioned group G3. Examples of the alkyl group in the alkyl group which may have a group selected from the above-mentioned group G3 include linear or branched C₁ to C₁₂ alkyl groups such as a methyl group, an ethyl group, a propyl group, an isopropyl group, a butyl group, an isobutyl group, a sec-butyl group, a tert-butyl group, a pentyl group, and a decyl group; and C₃ to C₁₂ cycloalkyl groups such as a cyclopropyl group, a 2,2-dimethylcyclopropyl group, a cyclopentyl group, a cyclohexyl group, and a menthyl group. Examples of the aryl group in the aryl group which may have a group selected from the above-mentioned group G3 include C₆ to C₁₀ aryl groups such as a phenyl group, a 2-methylphenyl group, a 4-methylphenyl group, and a naphthyl group.

R³ and R⁴ are preferably bonded together to form an optionally substituted divalent hydrocarbon group.

In the formula (2-1), examples of the alkyl groups in the optionally substituted alkyl group represented by R² and the optionally substituted alkyl group represented by R⁵ include linear or branched C₁ to C₁₂ alkyl groups such as a methyl group, an ethyl group, a propyl group, an isopropyl group, a butyl group, an isobutyl group, a sec-butyl group, a tert-butyl group, a pentyl group, and a decyl group; and C₃ to C₁₂ cycloalkyl groups such as a cyclopropyl group, a 2,2-dimethylcyclopropyl group, a cyclopentyl group, a cyclohexyl group, a menthyl group, and an adamantyl group.

Examples of the optional substituent of the alkyl group include groups selected from the following group G4:

### <Group G4>

a C₆ to C₁₀ aryl group which may have a C₁ to C₁₀ alkoxy group;

a C₁ to C₁₀ alkoxy group which may have a fluorine atom;

a C₇ to C₂₀ aralkyloxy group which may have a C₁ to C₁₀ alkoxy group;

a C₇ to C₂₀ aralkyloxy group which may have a C₆ to C₁₀ aryloxy group;

a C₆ to C₁₀ aryloxy group which may have a C₁ to C₁₃ alkoxy group;

a C₆ to C₁₀ aryloxy group which may have a C₆ to C₁₀ aryloxy group; and

a C₂ to C₁₀ acyl group which may have a C₁ to C₁₀ alkoxy group.

In the group G4, examples of the C₆ to C₁₀ aryl group which may have a C₁ to C₁₀ alkoxy group include a phenyl group, a naphthyl group, a 4-methylphenyl group, and a 4-methoxyphenyl group;

examples of the C₁ to C₁₀ alkoxy group which may have a fluorine atom include a methoxy group, an ethoxy group, a propoxy group, an isopropoxy group, a butoxy group, an isobutoxy group, a sec-butoxy group, a tert-butoxy group, and a trifluoromethoxy group;

examples of the C₇ to C₂₀ aralkyloxy group which may have a C₁ to C₁₀ alkoxy group include a benzyloxy group, a 4-methylbenzyloxy group, and a 4-methoxybenzyloxy group;

examples of the C₇ to C₂₀ aralkyloxy group which may have a C₆ to C₁₀ aryloxy group include a 3-phenoxybenzyloxy group;

examples of the C₆ to C₁₀ aryloxy group which may have a C₁ to C₁₀ alkoxy group include a phenoxy group, a 2-methylphenoxy group, a 4-methylphenoxy group, and a 4-methoxyphenoxy group;

examples of the C₆ to C₁₀ aryloxy group which may have a C₆ to C₁₀ aryloxy group include a 3-phenoxyphenoxy group; and

examples of the C₂ to C₁₀ acyl group which may have a C₁ to C₁₀ alkoxy group include an acetyl group, a propionyl group, a benzylcarbonyl group, a 4-methylbenzylcarbonyl group, a 4-methoxybenzylcarbonyl group, a benzoyl group, a 2-methylbenzoyl group, a 4-methylbenzoyl group, and a 4-methoxybenzoyl group.

Examples of the alkyl group having a group selected from the group G4 include a methoxymethyl group, an ethoxymethyl group, a methoxyethyl group, a benzyl group, a 4-fluorobenzyl group, a 4-methylbenzyl group, a phenoxymethyl group, a 2-oxopropyl group, a 2-oxobutyl group, and a phenacyl group.

In the formula (2-1), examples of the aryl group in the optionally substituted aryl group represented by R² and the optionally substituted aryl group represented by R⁵ include C₆ to C₂₀ aryl groups such as a phenyl group, a naphthyl group, a 2-methylphenyl group, a 4-methylphenyl group, a 2,6-dimethylphenyl group, a 2,4,6-trimethylphenyl group, and a 2,6-diisopropylphenyl group.

Examples of the optional substituent of the aryl group include groups selected from the following group G5:

### <Group G5>

a C₁ to C₁₀ alkoxy group which may have a fluorine atom or a C₁ to C₁₀ alkoxy group; and

a halogen atom.

In the group G5, examples of the C₁ to C₁₀ alkoxy group which may have a fluorine atom or a C₁ to C₁₀ alkoxy group include a methoxy group, an ethoxy group, a propoxy group, an isopropoxy group, a butoxy group, an isobutoxy group, a sec-butoxy group, a tert-butoxy group, a pentyloxy group, a cyclopentyloxy group, a fluoromethoxy group, a trifluoromethoxy group, a methoxymethoxy group, an ethoxymethoxy group, and a methoxyethoxy group; and examples of the halogen atom include a fluorine atom and a chlorine atom.

Examples of the aryl having a group selected from the group G5 include a 4-chlorophenyl group, a 4-methoxyphenyl group, and a 2,6-dichlorophenyl group.

In the formula (2-1), R⁵ may be bonded together with R⁴ to form an optionally substituted divalent hydrocarbon group. Examples of the divalent hydrocarbon group include polymethylene groups such as an ethylene group, a trimethylene group, and a tetramethylene group; a vinylene group, a propane-1,2-diyl group, a propene-1,2-diyl group, a butane-1,2-diyl group, a 2-butene-1,2-diyl group, a cyclopentane-1,2-diyl group, a cyclohexane-1,2-diyl group, and an o-phenylene group. Examples of the optional substituent of the divalent hydrocarbon group include groups selected from the above-mentioned group G3.

In the formula (2-1), examples of the anion represented by X⁻ include halide ions such as a chloride ion, a bromide ion, and an iodide ion; alkane sulfonate ions which may have a fluorine atom such as methane sulfonate and trifluoromethane sulfonate; acetate ions which may have a halogen atom such as trifluoroacetate and trichloroacetate; a nitrate ion; a perchlorate ion; tetrahaloborate ions such as tetrafluoroborate and tetrachloroborate; hexahalophosphate ions such as hexafluorophosphate; hexahaloantimonate ions such as hexafluoroantimonate and hexachlorcantimonate; pentahalostannate ions such as pentafluorostannate and pentachlorostannate; and optionally substituted tetraarylborates such as tetraphenylborate, tetrakis(pentafluorophenyl)borate, and tetrakis[3,5-bis(trifluoromethyl)phenyl]borate.

The compound (2-1) is preferably a compound represented by a formula (2-4)

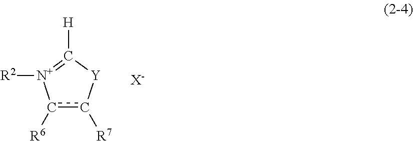

wherein, R², Y and X⁻ are the same as described above, respectively; R⁶ and R⁷ each independently represent a hydrogen atom, an optionally substituted alkyl group or an optionally substituted aryl group or R⁶ and R⁷ may be bonded together to form a ring together with carbon atoms to which R⁶ and R⁷ are bonded or R⁶ represents a hydrogen atom, an optionally substituted alkyl group or an optionally substituted aryl group and R⁷ may be bonded together with R⁵ to form an optionally substituted divalent hydrocarbon group, and

[symbol]

represents a single bond or a double bond (hereinafter, may be referred to as “compound (2-4)” in some cases), or a compound represented by a formula (2-5)

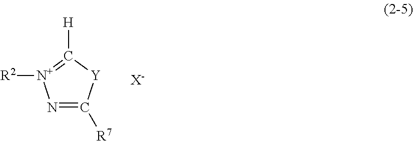

wherein, R², Y, and X⁻ are the same as described above, respectively; R⁷ represents a hydrogen atom, an optionally substituted alkyl group or an optionally substituted aryl group or R⁷ may be bonded together with R⁵ to form an optionally substituted divalent hydrocarbon group (hereinafter, may be referred to as “compound (2-5)” in some cases); and more preferably a compound (2-4).

Hereinafter, the compound (2-4) and the compound (2-5) will be described.

In the formula (2-4) and the formula (2-5), R² is the same as R² in the formula (2-1) and Y is the same as Y in the formula (2-1). In the case where Y in the formula (2-4) and the formula (2-5) is a group represented by —N(R⁵)—, R⁵ is the same as R⁵ in the formula (2-1). In the formula (2-4) and the formula (2-5), X⁻ is the same as X⁻ in the formula (2-1).

In the formula (2-4), R² is preferable to be a bulky group, and in the case Y is a group represented by —N(R⁵)—, it is preferable that either one of R² and R⁵ is a bulky group, and it is more preferable that both of R² and R⁵ are bulky groups. R² and R⁵ may be the same groups or different groups.

Examples of the bulky group for R² and R⁵ include C₄ to C₁₂ tertiary alkyl groups such as a tert-butyl group and a tert-pentyl group; C₃ to C₁₀ cycloalkyl groups such as a cyclopropyl group, a 2,2-dimethylcyclopropyl group, a cyclopentyl group, a cyclohexyl group, a menthyl group, and an adamantyl group; phenyl groups having substituents at least at the 2nd and 6th positions (2,6-di-substituted phenyl group) such as a 2,6-dimethylphenyl group, a 2,6-dichlorophenyl group, a 2,6-dibromophenyl group, a 2,4,6-trimethylphenyl group, and a 2,6-diisopropylphenyl group; and a naphthyl group having a C₁ to C₁₀ alkyl group at the 2nd position such as a 2-methylnaphthyl group. Examples of the substituent of the 2,6-di-substituted phenyl group include a C₁ to C₁₂ alkyl group and a halogen atom.

The bulky group for R² and R⁵ is preferably a tert-butyl group, a tert-pentyl group, a cyclohexyl group, an adamantyl group, or a 2,6-di-substituted phenyl group; more preferably a 2,6-di-substituted phenyl group; and furthermore preferably a 2,6-di-bromophenyl group and a 2,6-di-isopropylphenyl group.

Examples of the alkyl group in the optionally substituted alkyl group represented by R⁶ in the formula (2-4) and the optionally substituted alkyl group represented by R⁷ in the formula (2-4) and the formula (2-5) include linear, branched, or cyclic C₁ to C₁₀ alkyl groups such as a methyl group, an ethyl group, a propyl croup, an isopropyl group, a butyl group, an isobutyl group, a sec-butyl group, a tert-butyl group, a pentyl group, a decyl group, a cyclopropyl group, a 2,2-dimethylcyclopropyl group, a cyclopentyl group, a cyclohexyl group, and a menthyl group.

Examples of the optional substituent of the alkyl group include groups selected from the above-mentioned group G3. Examples of the alkyl group having a group selected from the group G3 include a fluoromethyl group, a trifluoromethyl group, a methoxymethyl group, an ethoxymethyl group, a methoxyethyl group, a benzyl group, a 4-fluorobenzyl group, a 4-methylbenzyl group, a phenoxymethyl group, a 2-oxopropyl group, a 2-oxobutyl group, a phenacyl group, and a 2-carboxyethyl group.

Examples of the aryl group in the optionally substituted aryl group represented by R⁶ in the formula (2-4) and the optionally substituted aryl group represented by R⁷ in the formula (2-4) and the formula (2-5) include C₆ to C₁₀ aryl groups such as a phenyl group, a 2-methylphenyl group, a 4-methylphenyl group, and a naphthyl group.

Examples of the optional substituent of the aryl group include groups selected from the above-mentioned group G3.

Examples of the aryl group having a group selected from the group G3 include a 4-chlorophenyl group and a 4-methoxyphenyl group.

In the formula (2-4), R⁶ and R⁷ may be bonded together to form a ring together with carbon atoms to which R⁶ and R⁷ are bonded. Examples of the ring include a cyclopentane ring, a cyclohexane ring, and a benzene ring.

In the formula (2-4), R⁶ and R⁷ are each independently preferable to be a hydrogen atom or an optionally substituted alkyl group, and R⁵ and R⁷ are more preferable to be both a hydrogen atom.

In the formula (2-4) and the formula (2-5), in the case where Y is a group represented by —N(R⁵)—, R⁵ and R⁷ may be bonded together to form an optionally substituted divalent hydrocarbon group. Examples of the divalent hydrocarbon group include polymethylene groups such as an ethylene group, a trimethylene group, and a tetramethylene group; a vinylene group, a propane-1,2-diyl group, a propene-1,2-diyl group, a butane-1,2-diyl group, a 2-butene-1,2-diyl group, a cyclopentane-1,2-diyl group, a cyclohexane-1,2-diyl group, and an o-phenylene group. Examples of the optional substituent of the divalent hydrocarbon group include groups selected from the above-mentioned group G3.

In the formula (2-4), in the case where Y is a group represented by —N(R⁵)—,

[symbol]

is preferably a single bond and in the case where Y is a group represented by —S—, [symbol]
is preferably a double bond.

Examples of the compound (2-4) include a compound (2-4) in which Y is a group represented by —N(R⁵)— and R² and R⁵ are independently a C₄ to C₁₂ tertiary alkyl group, a C₃ to C₁₀ cycloalkyl group, a phenyl group having substituents at least at the 2nd and 6th positions, or a naphthyl group having a C₁ to C₁₀ alkyl group at the 2nd position;

a compound (2-4) in which Y is a group represented by —N(R⁵)—; R² and R⁵ are independently a C₄ to C₁₂ tertiary alkyl group, a C₃ to C₁₀ cycloalkyl group, a phenyl group having substituents at least at the 2nd and 6th positions, or a naphthyl group having a C₁ to C₁₀ alkyl group at the 2nd position; and [symbol]
is a single bond;
a compound (2-4) in which Y is a group represented by —N(R⁵)— and R² and R⁵ are independently a C₄ to C₁₂ tertiary alkyl group, a C₃ to C₁₀ cycloalkyl group, a phenyl group having a C₁ to C₁₂ alkyl group or a halogen atom at least at the 2nd and 6th positions, or a naphthyl group having a C₁ to C₁₀ alkyl group at the 2nd position;
a compound (2-4) in which Y is a group represented by —R² and R⁵ are independently a C₄ to C₁₂ tertiary alkyl group, a C₃ to C₁₀ cycloalkyl group, a phenyl group having a C₁ to C₁₂ alkyl group or a halogen atom at least at the 2nd and 6th positions, or a naphthyl group having a C₁ to C₁₀ alkyl group at the 2nd position; and [symbol]
is a single bond;
a compound (2-4) in which Y is a group represented by —N(R⁵)— and R² and R⁵ are independently a tert-butyl group, a tert-pentyl group, a cyclohexyl group, an adamantyl group, a 2,6-dimethylphenyl group, a 2,6-dichlorophenyl group, a 2,6-dibromophenyl group, a 2,4,6-trimethylphenyl group, or a 2,6-diisopropylphenyl group;
a compound (2-4) in which Y is a group represented by —N(R⁵)—; R² and R⁵ are independently a tert-butyl group, a tert-pentyl group, a cyclohexyl group, an adamantyl group, a 2,6-dimethylphenyl group, a 2,6-dichlorophenyl group, a 2,6-dibromophenyl group, a 2,4,6-trimethylphenyl group, or a 2,6-diisopropylphenyl group; and [symbol]
is a single bond;
a compound (2-4) in which Y is a group represented by —N(R⁵)—; R² and R⁵ are independently a C₄ to C₁₂ tertiary alkyl group, a C₃ to C₁₀ cycloalkyl group, a phenyl group having a C₁ to C₁₂ alkyl group or a halogen atom at least at the 2nd and 6th positions, or a naphthyl group having a C₁ to C₁₀ alkyl at the 2nd position; [symbol]
is a single bond; and R⁶ and R⁷ are independently a hydrogen atom or a linear, branched or cyclic C₁ to C₁₀ alkyl group which may have a group selected from the above-mentioned group G3;
a compound (2-4) in which Y is a group represented by —N(R⁵)—; R² and R⁵ are independently a C₄ to C₁₂ tertiary alkyl group, a C₃ to C₁₀ cycloalkyl group, a phenyl group having a C₁ to C₁₂ alkyl group or a halogen atom at least at the 2nd and 6th positions, or a naphthyl group having a C₁ to C₁₀ alkyl at the 2nd position; [symbol]
is a single bond; and R⁶ and R⁷ are a hydrogen atom;
a compound (2-4) in which Y is a group represented by —N(R⁵)— and R² and R⁵ are independently a tert-butyl group, a tert-pentyl group, a cyclohexyl group, an adamantyl group, a 2,6-dimethylphenyl group, a 2,6-dichlorophenyl group, a 2,6-dibromophenyl group, a 2,4,6-trimethylphenyl group, or a 2,6-diisopropylphenyl group; and
a compound (2-4) in which Y is a group represented by —N(R⁵)—; R² and R⁵ are independently a tert-butyl group, a tert-pentyl group, a cyclohexyl group, an adamantyl group, a 2,6-dimethylphenyl group, a 2,6-dichlorophenyl group, a 2,6-dibromophenyl group, a 2,4,6-trimethylphenyl group, or a 2,6-diisopropylphenyl group; [symbol]
is a single bond; and R⁶ and R⁷ are a hydrogen atom;
a compound (2-4) in which Y is a group represented by —S—; R² is a C₄ to C₁₂ tertiary alkyl group, a C₃ to C₁₀ cycloalkyl group, a phenyl group having substituents at least at the 2nd and 6th positions, or a naphthyl group having a C₁ to C₁₀ alkyl group at the 2nd position;
a compound (2-4) in which Y is a group represented by —S—; R² is a C₄ to C₁₂ tertiary alkyl group, a C₃ to C₁₀ cycloalkyl group, a phenyl group having substituents at least at the 2nd and 6th positions, or a naphthyl group having a C₁ to C₁₀ alkyl group at the 2nd position; and [symbol]
is a double bond;
a compound (2-4) in which Y is a group represented by —S— and R² is a C₄ to C₁₂ tertiary alkyl group, a C₃ to C₁₀ cycloalkyl group, a phenyl group having a C₁ to C₁₂ alkyl group or a halogen atom at least at the 2nd and 6th positions, or a naphthyl group having a C₁ to C₁₀ alkyl group at the 2nd position;
a compound (2-4) in which Y is a group represented by —S—; R² is independently a C₄ to C₁₂ tertiary alkyl group, a C₃ to C₁₀ cycloalkyl group, a phenyl group having a C₁ to C₁₂ alkyl group or a halogen atom at least at the 2nd and 6th positions, or a naphthyl group having a C₁ to C₁₀ alkyl group at the 2nd position; and [symbol]
is a double bond;

a compound (2-4) in which Y is a group represented by —S— and R² is a tert-butyl group, a tert-pentyl group, a cyclohexyl group, an adamantyl group, a 2,6-dimethylphenyl group, a 2,6-dichlorophenyl group, a 2,6-dibromophenyl group, a 2,4,6-trimethylphenyl group, or a 2,6-diisopropylphenyl group;

a compound (2-4) in which Y is a group represented by —S—; R² is a tert-butyl group, a tert-pentyl group, a cyclohexyl group, an adamantyl group, a 2,6-dimethylphenyl group, a 2,6-dichlorophenyl group, a 2,6-dibromophenyl group, a 2,4,6-trimethylphenyl group, or a 2,6-diisopropylphenyl group; and [symbol]
is a double bond;
a compound (2-4) in which. Y is a group represented by —S—; R² is independently a C₄ to C₁₂ tertiary alkyl group, a C₃ to C₁₀ cycloalkyl group, a phenyl group having a C₁ to C₁₂ alkyl group or a halogen atom at least at the 2nd and 6th positions, or a naphthyl group having a C₁ to C₁₀ alkyl at the 2nd position; [symbol]
is a double bond; and R⁶ and R⁷ are independently a hydrogen atom or a linear, branched or cyclic C₁ to C₁₀ alkyl group which may have a group selected from the above-mentioned group G3;
a compound (2-4) in which Y is a group represented by —N(R⁵)—; R² and R⁵ are independently a tert-butyl group, a tert-pentyl group, a cyclohexyl group, an adamantyl group, a 2,6-dimethylphenyl group, a 2,6-dichlorophenyl group, a 2,6-dibromophenyl group, a 2,4,6-trimethylphenyl group, or a 2,6-diisopropylphenyl group; and R⁶ and R⁷ are independently a hydrogen atom, a methyl group, an ethyl group, a propyl group, an isopropyl group, a butyl group, an isobutyl group, a sec-butyl group, a tert-butyl group, a pentyl group, a decyl group, a cyclopropyl group, a 2,2-dimethylcyclopropyl group, a cyclopentyl group, a cyclohexyl group, or a menthyl group; and
a compound (2-4) in which Y is a group represented by —N(R⁵)—; R² and R⁵ are independently a tert-butyl group, a tert-pentyl group, a cyclohexyl group, an adamantyl group, a 2,6-dimethylphenyl group, a 2,6-dichlorophenyl group, a 2,6-dibromophenyl group, a 2,4,6-trimethylphenyl group, or a 2,6-diisopropylphenyl group; [symbol]
is a single bond; R⁶ and R⁷ are independently a hydrogen atom, a methyl group, an ethyl group, a propyl group, an isopropyl group, a butyl group, an isobutyl group, a sec-butyl group, a tert-butyl group, a pentyl group, a decyl group, a cyclopropyl group, a 2,2-dimethylcyclopropyl group, a cyclopentyl group, a cyclohexyl group, or a menthyl group.

Examples of the compound (2-4) include 1,3-di-tert-butylimidazolium chloride, 1,3-di-tert-butylimidazolinium chloride, 1,3-dicyclohexylimidazolium chloride, 1,3-dicyclohexylimidazolinium chloride, 1,3-diadamantylimidazolium chloride, 1,3-diadamantylimidazolinium chloride, 1,3-diphenylimidazolium chloride, 1,3-diphenylimidazolinium chloride, 1,3-bis[(2,6-diisopropyl)phenyl]imidazolium chloride, 1,3-bis[(2,6-diisopropyl)phenyl]imidazolinium chloride, 1,3-bis[(2,4,6-trimethyl)phenyl]imidazolium chloride, 1,3-bis[(2,4,6-trimethyl)phenyl]imidazolinium chloride, 1,3-bis[(2,6-dibromo)phenyl]imidazolinium chloride, 1,3-bis[(2,4,6-tribromo)phenyl]imidazolinium chloride, 4,5-dimethyl-1,3-bis[(2,4,6-trimethyl)phenyl]imidazolium chloride, 4,5-dimethyl-1,3-bis[(2,4,6-trimethyl)phenyl]imidazolinium chloride, 4,5-dimethyl-1,3-bis[(2,6-diisopropyl)phenyl]imidazolium chloride, 4,5-dimethyl-1,3-bis[(2,6-diisopropyl)phenyl]imidazolinium chloride, 4,5-dichloro-1,3-bis[(2,6-diisopropyl)phenyl]imidazolium chloride, 4,5-dichloro-1,3-bis[(2,6-diisopropyl)phenyl]imidazolinium chloride, 4,5-diphenyl-1,3-bis[(2,4,6-trimethyl)phenyl]imidazolium chloride, 4,5-diphenyl-1,3-bis[(2,4,6-trimethyl)phenyl]imidazolinium chloride, 4,5-difluoro-1,3-bis[(2,6-diisopropyl)phenyl]imidazolium chloride, 4,5-difluoro-1,3-bis[(2,6-diisopropyl)phenyl]imidazolinium chloride, 4-methyl-1,3-bis[(2,4,6-trimethyl)phenyl]imidazolium chloride, 4-methyl-1,3-bis[(2,4,6-trimethyl)phenyl]imidazolinium chloride, 1,3-bis[(2,6-dichloro)phenyl]imidazolium chloride, 1,3-bis[(2,6-dichloro)phenyl]imidazolinium chloride, 1-tert-butyl-3-phenylimidazolium chloride, 1-tert-butyl-3-phenylimidazolinium chloride, 1-cyclohexyl-3-[(2,6-diisopropyl)phenyl]imidazolium chloride, 1-cyclohexyl-3-[(2,6-diisopropyl)phenyl]imidazolinium chloride, 1-phenyl-3-[(2,4,6-trimethyl)phenyl]imidazolium chloride, 1-phenyl-3-[(2,4,6-trimethyl)phenyl]imidazolinium chloride, 1-tert-butyl-3-[(2,6-diisopropyl)phenyl]imidazolium chloride, 1-tert-butyl-3-[(2,6-diisopropyl)phenyl]imidazolinium chloride, 1-tert-butyl-3-[(2,4,6-trimethyl)phenyl]imidazolium chloride, 1-tert-butyl-3-[(2,4,6-trimethyl)phenyl]imidazolinium chloride, 3-ethylbenzothiazolium bromide, 3-butylbenzothiazolium chloride, 3-(2,6-diisopropyl)phenyl-4,5-dimethylthiazolium chloride, 3-phenyl-4,5-dimethylthiazolium chloride, 3-benzylthiazolium chloride, 3-benzyl-4-methylthiazolium chloride, 3-n-butyl-4-methylthiazolium chloride, 3-n-hexyl-4-methylthiazolium chloride, 3-cyclohexyl-4-methylthiazolium chloride, 3-n-octyl-4-methylthiazolium chloride, and 3-(2,4,6-trimethyl)phenyl-4,5-dimethylthiazolium chloride.

Example of the compound (2-5) include a compound (2-5) in which Y is a group represented by —N(R⁵)—; R² is a linear or branched C₁ to C₁₂ alky group or a C₆ to C₂₀ aryl group; and R⁵ is a linear or branched C₁ to C₁₂ alky group or a C₆ to C₂₀ aryl group;

a compound (2-5) in which Y is a group represented by —N(R⁵)—; R² represents a linear or branched C₁ to C₁₂ alky group or a C₆ to C₂₀ aryl group; R⁵ is a linear or branched C₁ to C₁₂ alky group or a C₆ to C₂₀ aryl group; and R⁷ is a C₁ to C₁₀ alky group or a C₆ to C₁₀ aryl group;
a compound (2-5) in which Y is a group represented by —S— and R² is a linear or branched C₁ to C₁₂ alky group or a C₆ to C₂₀ aryl group; and
a compound (2-5) in which Y is a group represented by —S—; R² is a linear or branched C₁ to C₁₂ alky group or a C₆ to C₂₀ aryl group; and R⁷ is a C₁ to C₁₀ alky group or a C₆ to C₁₀ aryl group.

Examples of the compound (2-3) include 1,4-dimethyl-1H-1,2,4-triazol-4-ium chloride, 1,3,4-triphenyl-1H-1,2,4-triazol-4-ium chloride, 3,5-diphenyl-1,3,4-thiadiazolium chloride, 3-methyl-5-phenyl-1,3,4-thiadiazolium chloride, and 6,7-dihydro-2-pentafluorophenyl-5H-pyrrolo[2,1-c]-1,2,4-triazolium chloride.

Examples thereof include a compound (2-4) and a compound (2-5) obtained by replacing “chloride” in these compound (2-4) and compound (2-5) with “iodide”, “bromide”, “methanesulfonate”, “trifluoromethanesulfonate”, “nitrate”, “perchlorate”, “tetrafluoroborate”, “tetrachloroborate”, “hexafluorophosphate”, “hexafluoroantimonate”, “hexachloroantimonate”, “pentafluorostannate”, “pentachlorostannate”, “tetraphenylborate”, “tetrakis(pentafluorophenyl)borate”, and “tetrakis[3,5-bis(trifluoromethyl)phenyl]borate”.

The compound (2-1) may be a commercialized product or may be produced according to the methods disclosed in, for example, J. Organometallic. Chem. Soc., 606, 49 (2000) and J. Org. Chem. Soc., 73, 2784 (2008).

The base to be reacted with the compound (2-1) in the step A is preferably at least one base selected from the group consisting of an organic base and an alkali metal alkoxide.

Examples of the organic base include tertiary amines such as triethylamine, trioctylamine, diisopropylethylamine, and 4-dimethylaminopyridine; nitrogen-containing aliphatic cyclic compounds such as 1,8-diazabicyclo[5,4,0]-7-undecene and 1,5,7-triazabicyclo[4,4,0]-5-decene; and nitrogen-containing aromatic compounds such as pyridine and imidazole.

Examples of the alkali metal of the alkali metal alkoxide include lithium, sodium, and potassium. Examples of the alkoxide include methoxide, ethoxide, n-propoxide, isopropoxide, tert-butoxide, and sec-butoxide. The alkali metal alkoxide is preferably at least one selected from the group consisting of lithium alkoxide, sodium alkoxide, and potassium alkoxide.

The alkali metal alkoxide may be a high purity product or in the form of an alcohol solution. In this case, an alcohol solvent to be contained in the alcohol solution is preferably the same as that to be used in the step A in terms of obtaining a 4-methylthio-2-oxo-butanoic acid ester with high purity.

The use amount of the base to be reacted with the compound (2-1) in the step A is, for example, in a range of 0.1 to 10 mol and preferably in a range of 0.5 to 3 mol per 1 mol of the compound (2-1).

Hereinafter, a method for generating a carbene catalyst by causing a reaction of the compound (2-1) and the base will be described.

The generation of the carbene catalyst may be carried out simultaneously with the oxidization reaction in the step A as described below or the carbene catalyst is generated previously and then the carbene catalyst is added to the oxidization reaction in the step A.

In the case of generating the carbene catalyst simultaneously with the oxidization reaction in the step A, a solvent may be used or may not be used. In the case of previously generating the carbene catalyst, the carbene catalyst is preferable to be generated in the presence of a solvent. The solvent preferably used is a solvent that does not react with the generated carbene catalyst and examples thereof include ether solvents such as tetrahydrofuran, methyl tert-butyl ether, cyclopentyl methyl ether, and diisopropyl ether; ester solvents such as ethyl acetate and butyl acetate; aromatic solvents such as toluene and chlorobenzene; nitrile solvents such as acetonitrile and propionitrile; and their solvent mixtures.

The use amount of the solvent is not limited and practically, for example, it is 100 parts by weight or less per 1 part by weight of the compound (2-1).

The mixing order of reaction reagents is not limited in the generation of the carbene catalyst. Examples of a preferable embodiment include methods of mixing the compound (2-1) and the solvent and adding the base to the mixture to be obtained.

The generation of the carbene catalyst is carried out in any condition of reduced pressure, normal pressure, and applied pressure and preferably carried out in a condition of normal pressure or applied pressure.

The reaction temperature in the generation of the carbene catalyst is different depending on the type of the compound (2-1), the type of the base, the use amount, the type of the carbene catalyst to be generated and the like, but is preferably in a range of −20° C. to 100° C. and more preferably in a range of 0° C. to 50° C. In the case where the reaction temperature is lower than −20° C., the generation rate of the carbene catalyst tends to be low and in the case where the reaction temperature is higher than 100° C., the carbene catalyst to be produced tends to be decomposed.

The progression of the reaction in the generation of the carbene catalyst can be confirmed by an analysis means, for example, thin-layer chromatography, nuclear magnetic resonance spectroscopic analysis, infrared absorption spectrometry, etc.

After completion of the reaction in the generation of the carbene catalyst, the reaction solution containing the carbene catalyst can be used as it is for the oxidation reaction of the step A and for the production of the compound (2-2) and the compound (2-3) described below. Further, after a salt produced in the reaction with the base to be used is removed by filtration if necessary, the reaction mixture to be obtained may be, for example, subjected to a concentration treatment if necessary and subsequently subjected to a cooling treatment or the like to take the carbene catalyst cut.

Next, the compound (2-2) and the compound (2-3) will be described.

The compound (2-2) is obtained by causing a reaction of the compound, which is obtained by causing a reaction of the compound (2-1) and the base, and an alcohol represented by R⁸OH. The compound (2-3) is obtained by causing a reaction of the compound, which is obtained by causing a reaction of the compound (2-1) and the base, and carbon dioxide. That is, R², R³, R⁴, and Y in the formula (2-2) and the formula (2-3) are respectively the same as those defined in the formula (2-1).

In the formula (2-2), examples of the alkyl group represented by R⁸ include linear or branched C₁ to C₆ alkyl groups such as a methyl group, an ethyl group, a propyl group, an isopropyl group, a butyl group, an isobutyl group, a sec-butyl group, a pentyl group, and a hexyl group.

The compound represented by the formula (2-2) is preferably a compound represented by a formula (2-6)

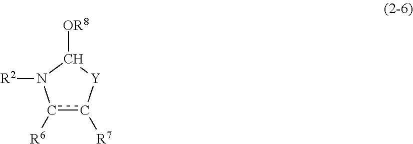

wherein, R², R⁸, R⁶, R⁷, and Y are the same as described above, respectively; and [symbol]
represents a single bond or a double bond
(hereinafter, may be referred to as “compound (2-6)” in some cases), or
a compound represented by a formula (2-7)

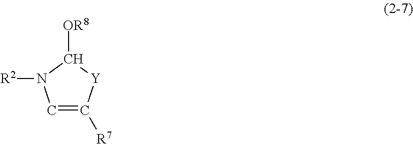

wherein, R², R⁷, R⁸, and Y are the same as described above, respectively
(hereinafter, may be referred to as “compound (2-7)” in some cases); and more preferably a compound (2-6).

The compound represented by the formula (2-3) is preferably a compound represented by a formula (2-8)

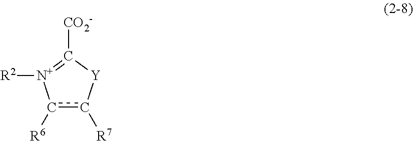

wherein, R², R⁶, R⁷, Y, and [symbol]
are the same as defined above, respectively
(hereinafter, may be referred to as “compound (2-8)” in some cases), or
a compound represented by a formula (2-9)

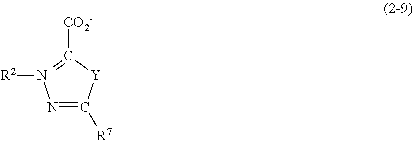

wherein, R², R⁷, and Y are the same as described above, respectively
(hereinafter, may be referred to as “compound (2-9)” in some cases); and more preferably a compound (2-8).

Hereinafter, the compound (2-6), the compound (2-7), the compound (2-8) and the compound (2-9) will be described.

In the formula (2-6), the formula (2-7), the formula (2-8), and the formula (2-9), R² is the same as R² in the formula (2-1) and Y is the same as Y in the formula (2-1). In the case where Y is a group represented by —N(R⁵)—, R⁵ is the same as R⁵ in the formula (2-1).

In the formula (2-6) and the formula (2-7), R⁸ is the same as R⁸ in the formula (2-2).

In the formula (2-6), the formula (2-7), the formula (2-8), and the formula (2-9), Y is preferably a group represented by —N(R⁵)—.

In the formula (2-6), the formula (2-7), the formula (2-8), and the formula (2-9), it is preferable that at least one of R² and R⁵ is a bulky group, and it is more preferable that both of R² and R⁵ are bulky groups. R² and R⁵ may be the same groups or different groups.

Examples of the bulky group for R² and R⁵ include

C₄ to C₁₂ tertiary alkyl groups such as a tert-butyl group and a tert-pentyl group;

C₃ to C₁₀ cycloalkyl groups such as a cyclopropyl group, a 2,2-dimethylcyclopropyl group, a cyclopentyl group, a cyclohexyl group, a menthyl group, and an adamantyl group;

phenyl groups having substituents at least at the 2nd and 6th positions (2,6-di-substituted phenyl group) such as a 2,6-dimethylphenyl group, a 2,6-dichlorophenyl group, a 2,4,6-trimethylphenyl group, and a 2,6-diisopropylphenyl group; and
a naphthyl group having a C₁ to C₁₀ alkyl group at the 2nd position such as a 2-methylnaphthyl group. Examples of the substituent in the 2,6-di-substituted phenyl group include a C₁ to C₁₂ alkyl group and a halogen atom.

The bulky group is preferably a tert-butyl group, a tert-pentyl group, a cyclohexyl group, an adamantyl group, or a 2,6-di-substituted phenyl group; more preferably a 2,6-di-substituted phenyl group; and furthermore preferably a 2,6-di-isopropylphenyl group.

In the formula (2-6) and the formula (2-8), R⁶ is the same as R⁶ in the formula (2-4); and in the formula (2-6), the formula (2-7), the formula (2-8), and the formula (2-9), R⁷ is the same as R⁷ in the formula (2-5).

In the formula (2-6) and the formula (2-8), R⁶ and R⁷ are each independently preferable to be a hydrogen atom or an optionally substituted alkyl group, and R⁶ and R⁷ are more preferable to be both a hydrogen atom.

In the formula (2-6) and the formula (2-8),

[symbol]

is preferably a single bond.

Examples of the compound (2-6) include a compound (2-6) in which Y is a group represented by —N(R⁵)— and R² and R⁵ are independently a C₄ to C₁₂ tertiary alkyl group, a C₃ to C₁₀ cycloalkyl group, a phenyl group having substituents at least at the 2nd and 6th positions, or a naphthyl group having a C₁ to C₁₀ alkyl group at the 2nd position;

a compound (2-6) in which Y is a group represented by —N(R⁵)—; R² and R⁵ are independently a C₄ to C₁₂ tertiary alkyl group, a C₃ to C₁₀ cycloalkyl group, a phenyl group having substituents at least at the 2nd and 6th positions, or a naphthyl group having a C₁ to C₁₀ alkyl group at the 2nd position; and [symbol]
is a single bond;
a compound (2-6) in which Y is a group represented by —N(R⁵)— and R² and R⁵ are independently a C₄ to C₁₂ tertiary alkyl group, a C₃ to C₁₀ cycloalkyl group, a phenyl group having a C₁ to C₁₂ alkyl group or a halogen atom at least at the 2nd and 6th positions, or a naphthyl group having a C₁ to C₁₀ alkyl group at the 2nd position;
a compound (2-6) in which Y is a group represented by —N(R⁵)—; R² and R⁵ are independently a C₄ to C₁₂ tertiary alkyl group, a C₃ to C₁₀ cycloalkyl group, a phenyl group having a C₁ to C₁₂ alkyl group or a halogen atom at least at the 2nd and 6th positions, or a naphthyl group having a C₁ to C₁₀ alkyl group at the 2nd position; and [symbol]
is a single bond;
a compound (2-6) in which Y is a group represented by —N(R⁵)— and R² and R⁵ are independently a tert-butyl group, a tert-pentyl group, a cyclohexyl group, an adamantyl group, a 2,6-dimethylphenyl group, a 2,6-dichlorophenyl group, a 2,6-dibromophenyl group, a 2,4,6-trimethylphenyl group, or a 2,6-diisopropylphenyl group;

a compound (2-6) in which Y is a group represented by —N(R⁵)—; R² and R⁵ are independently a tert-butyl group, a tert-pentyl group, a cyclohexyl group, an adamantyl group, a 2,6-dimethylphenyl group, a 2,6-dichlorophenyl group, a 2,6-dibromophenyl group, a 2,4,6-trimethylphenyl group, or a 2,6-diisopropylphenyl group; and

[symbol]

is a single bond;
a compound (2-6) in which Y is a group represented by —N(R⁵)—; R² and R⁵ are independently a C₄ to C₁₂ tertiary alkyl group, a C₃ to C₁₀ cycloalkyl group, a phenyl group having a C₁ to C₁₂ alkyl group or a halogen atom at least at the 2nd and 6th positions, or a naphthyl group having a C₁ to C₁₀ alkyl at the 2nd position; [symbol]
is a single bond; and R⁶ and R⁷ are independently a hydrogen atom or a C₁ to C₁₀ linear, branched or cyclic alkyl group which may have a group selected from the above-mentioned group G3;
a compound (2-6) in which Y is a group represented by —N(R⁵)—; R² and R⁵ are independently a C₄ to C₁₂ tertiary alkyl group, a C₃ to C₁₀ cycloalkyl group, a phenyl group having a C₁ to C₁₂ alkyl group or a halogen atom at least at the 2nd and 6th positions, or a naphthyl group having a C₁ to C₁₀ alkyl at the 2nd position; [symbol]
is a single bond; and R⁶ and R⁷ are a hydrogen atom;
a compound (2-6) in which Y is a group represented by —N(R⁵)— and R² and R⁵ are independently a tort-butyl group, a tert-pentyl group, a cyclohexyl group, an adamantyl group, a 2,6-dimethylphenyl group, a 2,6-dichlorophenyl group, a 2,6-dibromophenyl group, a 2,4,6-trimethylphenyl group, or a 2,6-diisopropylphenyl group; and
a compound (2-6) in which Y is a group represented by —N(R⁵)—; R² and R⁵ are independently a tert-butyl group, a tert-pentyl group, a cyclohexyl group, an adamantyl group, a 2,6-dimethylphenyl group, a 2,6-dichlorophenyl group, a 2,6-dibromophenyl group, a 2,4,6-trimethylphenyl group, or a 2,6-diisopropylphenyl group; [symbol]
is a single bond; and R⁶ and R⁷ are a hydrogen atom;
a compound (2-6) in which Y is a group represented by —S—; R² is a C₄ to C₁₂ tertiary alkyl group, a C₃ to C₁₀ cycloalkyl group, a phenyl group having substituents at least at the 2nd and 6th positions, or a naphthyl group having a C₁ to C₁₀ alkyl group at the 2nd position;
a compound (2-6) in which Y is a group represented by —S—; R² is a C₄ to C₁₂ tertiary alkyl group, a C₃ to C₁₀ cycloalkyl group, a phenyl group having substituents at least at the 2nd and 6th positions, or a naphthyl group having a C₁ to C₁₀ alkyl group at the 2nd position; and [symbol]
is a double bond;
a compound (2-6) in which Y is a group represented by —S— and R² is a C₄ to C₂₂ tertiary alkyl group, a C₃ to C₁₀ cycloalkyl group, a phenyl group having a C₁ to C₁₂ alkyl group or a halogen atom at least at the 2nd and 6th positions, or a naphthyl group having a C₁ to C₁₀ alkyl group at the 2nd position;
a compound (2-6) in which Y is a group represented by —S—; R² is independently a C₄ to C₁₂ tertiary alkyl group, a C₃ to C₁₀ cycloalkyl group, a phenyl group having a C₁ to C₁₂ alkyl group or a halogen atom ac least at the 2nd and 6th positions, or a naphthyl group having a C₁ to C₁₀ alkyl group at the 2nd position; and [symbol]
is a double bond;
a compound (2-6) in which Y is a group represented by —S— and R² is a tert-butyl group, a tert-pentyl group, a cyclohexyl group, an adamantyl group, a 2,6-dimethylphenyl group, a 2,6-dichlorophenyl group, a 2,6-dibromophenyl group, a 2,4,6-trimethylphenyl group, or a 2,6-diisopropylphenyl group;
a compound (2-6) in which Y is a group represented by —S—; R² is a tert-butyl group, a tert-pentyl group, a cyclohexyl group, an adamantyl group, a 2,6-dimethylphenyl group, a 2,6-dichlorophenyl group, a 2,6-dibromophenyl group, a 2,4,6-trimethylphenyl group), or a 2,6-diisopropylphenyl group; and [symbol]
is a double bond;
a compound (2-6) in which Y is a group represented by —S—; R² is independently a C₄ to C₁₂ tertiary alkyl group, a C₃ to C₁₀ cycloalkyl group, a phenyl group having a C₁ to C₁₂ alkyl group or a halogen atom at least at the 2nd and 6th positions, or a naphthyl group having a C₁ to C₁₀ alkyl at the 2nd position; [symbol]
is a double bend; and R⁶ and R⁷ are independently a hydrogen atom or a C₁ to C₁₀ linear, branched or cyclic alkyl group which may have a group selected from the above-mentioned group G3;
a compound (2-6) in which Y is a group represented by —N(R⁵)—; R² and R⁵ are independently a tert-butyl group, a tert-pentyl group, a cyclohexyl group, an adamantyl group, a 2,6-dimethylphenyl group, a 2,6-dichlorophenyl group, a 2,6-dibromophenyl group, a 2,4,6-trimethylphenyl group, or a 2,6-diisopropylphenyl group; and R⁶ and R⁷ are independently a hydrogen atom, a methyl group, an ethyl group, a propyl group, an isopropyl group, a butyl group, an isobutyl group, a sec-butyl group, a tert-butyl group, a pentyl group, a decyl group, a cyclopropyl group, a 2,2-dimethylcyclopropyl group, a cyclopentyl group, a cyclohexyl group, or a menthyl group; and
a compound (2-6) in which Y is a group represented by —N(R⁵)—; R² and R⁵ are independently a tert-butyl group, a tert-pentyl group, a cyclohexyl group, an adamantyl group, a 2,6-dimethylphenyl group, a 2,6-dichlorophenyl group, a 2,6-dibromophenyl group, a 2,4,6-trimethylphenyl group, or a 2,6-diisopropylphenyl group; [symbol]
is a single bond; R⁶ and R⁷ are independently a hydrogen atom, a methyl group, an ethyl group, a propyl group, an isopropyl group, a butyl group, an isobutyl group, a sec-butyl group, a tert-butyl group, a pentyl group, a decyl group, a cyclopropyl group, a 2,2-dimethylcyclopropyl group, a cyclopentyl group, a cyclohexyl group, or a menthyl group.

Examples of the compound (2-6) include 2-methoxy-1,3-di-tert-butylimidazolidine, 2-ethoxy-1,3-di-tert-butylimidazolidine, 2-n-propoxy-1,3-di-tert-butylimidazolidine, 2-methoxy-1,3-dicyclohexylimidazolidine, 2-ethoxy-1,3-dicyclohexylimidazolidine, 2-propoxy-1,3-dicyclohexylimidazolidine, 2-methoxy-1,3-diadamantylimidazolidine, 2-methoxy-1,3-diphenyimidazolidine, 2-methoxy-1,3-bis[(2,6-diisopropyl)phenyl]imidazolidine, 2-methoxy-1,3-bis[(2,4,6-trimethyl)phenyl]imidazolidine, 2-ethoxy-1,3-bis[(2,6-diisopropyl)phenyl]imidazolidine, 2-ethoxy-1,3-bis[(2,4,6-trimethyl)phenyl]imidazolidine, 2-propoxy-1,3-bis[(2,6-diisopropyl)phenyl]imidazolidine, 2-propoxy-1,3-bis[(2,4,6-trimethyl)phenyl]imidazolidine, 2-butoxy-1,3-bis[(2,6-diisopropyl)phenyl]imidazolidine, 2-butoxy-1,3-bis[(2,4,6-trimethyl)phenyl]imidazolidine, 2-isopropoxy-1,3-bis[(2,6-diisopropyl)phenyl]imidazolidine, 2-isopropoxy-1,3-bis[(2,4,6-trimethyl)phenyl]imidazolidine, 2-methoxy-4,5-dimethyl-1,3-bis[(2,6-diisopropyl)phenyl]imidazolidine, 2-methoxy-4,5-dimethyl-1,3-bis[(2,4,6-trimethyl)phenyl]imidazolidine, 2-ethoxy-4,5-dimethyl-1,3-bis[(2,6-diisopropyl)phenyl]imidazolidine, 2-ethoxy-4,5-dimethyl-1,3-bis[(2,4,6-trimethyl)phenyl]imidazolidine, 2-methoxy-4,5-dichloro-1,3-bis[(2,6-diisopropyl)phenyl]imidazolidine, 2-methoxy-4,5-diphenyl-1,3-bis[(2,4,6-trimethyl)phenyl]imidazolidine, 2-methoxy-4,5-difluoro-1,3-bis[(2,6-diisopropyl)phenyl]imidazolidine, 2-methoxy-4-methyl-1,3-bis[(2,4,6-trimethyl)phenyl]imidazolidine, 2-methoxy-1,3-bis[(2,6-dichloro)phenyl]imidazolidine, 2-methoxy-1-tert-butyl-3-phenylimidazolidine, 2-methoxy-1-cyclohexyl-3-[(2,6-diisopropyl)phenyl]imidazolidine, 2-methoxy-1-phenyl-3-[(2,4,6-trimethyl)phenyl]imidazolidine, 2-ethoxy-1-tert-butyl-3-[(2,6-diisopropyl)phenyl]imidazolidine, and 2-ethoxy-1-tert-butyl-3-[(2,4,6-trimethyl)phenyl]imidazolidine.

Example of the compound (2-7) include

a compound (2-7) in which Y is a group represented by —N(R⁵)—; R² is a linear or branched C₁ to C₁₂ alky group or a C₆ to C₂₀ aryl group; and R⁵ is a linear or branched C₁ to C₁₂ alky group or a C₆ to C₂₀ aryl group;
a compound (2-7) in which Y is a group represented by —N(R⁵)—; R² represents a linear or branched C₁ to C₁₂ alky group or a C₆ to C₂₀ aryl group; R⁵ is a linear or branched C₁ to C₁₂ alky group or a C₆ to C₂₀ aryl group; and R⁷ is a C₁ to C₁₀ alky group or a C₆ to C₁₀ aryl group;
a compound (2-7) in which Y is a group represented by —S— and R² is a linear or branched C₁ to C₁₂ alky group or a C₆ to C₂₀ aryl group; and
a compound (2-7) in which Y is a group represented by —S—; R² is a linear or branched C₁ to C₁₂ alky group or a C₆ to C₂₀ aryl group; and R⁷ is a C₁ to C₁₀ alky group or a C₅ to C₁₀ aryl group.

Examples of the compound (2-7) include 5-methoxy-1,4-dimethyl-1,2,4(5H)-triazoline, and 5-methoxy-1,3,4-triphenyl-1,2,4(5H)-triazoline.

Examples of the compound (2-8) include a compound (2-8) in which Y is a group represented by —N(R⁵)— and R² and R⁵ are independently a C₄ to C₂₂ tertiary alkyl group, a C₃ to C₁₀ cycloalkyl group, a phenyl group having substituents at least at the 2nd and 6th positions, or a naphthyl group having a C₁ to C₁₀ alkyl group at the 2nd position;

a compound (2-8) in which Y is a group represented by —N(R⁵)—; R² and R⁵ are independently a C₄ to C₁₂ tertiary alkyl group, a C₃ to C₁₀ cycloalkyl group, a phenyl group having substituents at least at the 2nd and 6th positions, or a naphthyl group having a C₁ to C₁₀ alkyl group at the 2nd position; and [symbol]
is a single bond;
a compound (2-8) in which Y is a group represented by —N(R⁵)— and R² and R⁵ are independently a C₄ to C₁₂ tertiary alkyl group, a C₃ to C₁₀ cycloalkyl group, a phenyl group having a C₁ to C₁₂ alkyl group or a halogen atom at least the 2nd and 6th positions, or a naphthyl group having a C₁ to C₁₀ alkyl group at the 2nd position;
a compound (2-8) in which Y is a group represented by —N(R⁵)—; R² and R⁵ are independently a C₄ to C₁₂ tertiary alkyl group, a C₃ to C₁₀ cycloalkyl group, a phenyl group having a C₁ to C₁₂ alkyl group or a halogen atom at least at the 2nd and 6th positions, or a naphthyl group having a C₁ to C₁₀ alkyl group at the 2nd position; and [symbol]
is a single bond;
a compound (2-8) in which Y is a group represented by —N(R⁵)— and R² and R⁵ are independently a tert-butyl group, a tert-pentyl group, a cyclohexyl group, an adamantyl group, a 2,6-dimethylphenyl group, a 2,6-dichlorophenyl group, a 2,6-dibromophenyl group, a 2,4,6-trimethylphenyl group, or a 2,6-diisopropylphenyl group;
a compound (2-8) in which Y is a group represented by —N(R⁵)—; R² and R⁵ are independently a tert-butyl group, a tert-pentyl group, a cyclohexyl group, an adamantyl group, a 2,6-dimethylphenyl group, a 2,6-dichlorophenyl group, a 2,6-dibromophenyl group, a 2,4,6-trimethylphenyl group, or a 2,6-diisopropylphenyl group; and [symbol]
is a single bond;
a compound (2-8) in which Y is a group represented by —N(R⁵)—; R² and R⁵ are independently a C₄ to C₁₂ tertiary alkyl group, a C₃ to C₁₀ cycloalkyl group, a phenyl group having a C₁ to C₁₂ alkyl group or a halogen atom at least at the 2nd and 6th positions, or a naphthyl group having a C₁ to C₁₀ alkyl at the 2nd position; [symbol]
is a single bond; and R⁶ and R⁷ are independently a hydrogen atom or a linear, branched or cyclic C₁ to C₁₀ alkyl group which may have a group selected from the above-mentioned group G3;
a compound (2-8) in which Y is a group represented by —N(R⁵)—; R² and R⁵ are independently a C₄ to C₁₂ tertiary alkyl group, a C₃ to C₁₀ cycloalkyl group, a phenyl group having a C₁ to C₁₂ alkyl group or a halogen atom at least at the 2nd and 6th positions, or a naphthyl group having a C₁ to C₁₀ alkyl at the 2nd position; [symbol]
is a single bond; and R⁶ and R⁷ are a hydrogen atom;
a compound (2-8) in which Y is a group represented by —N(R⁵)— and R² and R⁵ are independently a tert-butyl group, a tert-pentyl group, a cyclohexyl group, an adamantyl group, a 2,6-dimethylphenyl group, a 2,6-dichlorophenyl group, a 2,6-dibromophenyl group, a 2,4,6-trimethylphenyl group, or a 2,6-diisopropylphenyl group; and
a compound (2-8) in which Y is a group represented by —N(R⁵)—; R² and R⁵ are independently a tert-butyl group, a tert-pentyl group, a cyclohexyl group, an adamantyl group, a 2,6-dimethylphenyl group, a 2,6-dichlorophenyl group, a 2,6-dibromophenyl group, a 2,4,6-trimethylphenyl group, or a 2,6-diisopropylphenyl group; [symbol]
is a single bond; and R⁶ and R⁷ are a hydrogen atom;
a compound (2-8) in which Y is a group represented by —S—; R² is a C₁ to C₁₂ tertiary alkyl group, a C₃ to C₁₀ cycloalkyl group, a phenyl group having substituents at least at the 2nd and 6th positions, or a naphthyl group having a C₁ to C₁₀ alkyl group at the 2nd position;
a compound (2-8) in which Y is a group represented by —S—; R² is a C₄ to C₁₂ tertiary alkyl group, a C₃ to C₁₀ cycloalkyl group, a phenyl group having substituents at least at the 2nd and 6th positions, or a naphthyl group having a C₁ to C₁₀ alkyl group at the 2nd position; and [symbol]
is a double bond;
a compound (2-8) in which Y is a group represented by —S— and R² is a C₄ to C₁₂ tertiary alkyl group, a C₃ to C₁₀ cycloalkyl group, a phenyl group having a C₁ to C₁₂ alkyl group or a halogen atom at least at the 2nd and 6th positions, or a naphthyl group having a C₁ to C₁₀ alkyl group at the 2nd position;
a compound (2-8) in which Y is a group represented by —S—; R² is independently a C₄ to C₁₂ tertiary alkyl group, a C₃ to C₁₀ cycloalkyl group, a phenyl group having a C₁ to C₁₂ alkyl group or a halogen atom at least at the 2nd and 6th positions, or a naphthyl group having a C₁ to C₁₀ alkyl group at the 2nd position; and [symbol]
is a double bond;
a compound (2-8) in which Y is a group represented by —S— and R² is a tert-butyl group, a tert-pentyl group, a cyclohexyl group, an adamantyl group, a 2,6-dimethylphenyl group, a 2,6-dichlorophenyl group, a 2,6-dibromophenyl group, a 2,4,6-trimethylphenyl group, or a 2,6-diisopropylphenyl group;
a compound (2-8) in which Y is a group represented by —S—; R² is a tert-butyl group, a tert-pentyl group, a cyclohexyl group, an adamantyl group, a 2,6-dimethylphenyl group, a 2,6-dichlorophenyl group, a 2,6-dibromophenyl group, a 2,4,6-trimethylphenyl group, or a 2,6-diisopropylphenyl group; and [symbol]
is a double bond;
a compound (2-8) in which Y is a group represented by —S—; R² is independently a C₄ to C₁₂ tertiary alkyl group, a C₃ to C₁₀ cycloalkyl group, a phenyl group having a C₁ to C₁₂ alkyl group or a halogen atom at least at the 2nd and 6th positions, or a naphthyl group having a C₁ to C₁₀ alkyl at the 2nd position; [symbol]
is a double bond; and R⁶ and R⁷ are independently a hydrogen atom or a linear, branched or cyclic C₁ to C₁₀ alkyl group which may have a group selected from the above-mentioned group G3;
a compound (2-8) in which Y is a group represented by —N(R⁵)—; R² and R⁵ are independently a tert-butyl group, a tert-pentyl group, a cyclohexyl group, an adamantyl group, a 2,6-dimethylphenyl group, a 2,6-dichlorophenyl group, a 2,6-dibromophenyl group, a 2,4,6-trimethylphenyl group, or a 2,6-diisopropylphenyl group; and R⁶ and R⁷ are independently a hydrogen atom, a methyl group, an ethyl group, a propyl group, an isopropyl group, a butyl group, an isobutyl group, a sec-butyl group, a tert-butyl group, a pentyl group, a decyl group, a cyclopropyl group, a 2,2-dimethylcyclopropyl group, a cyclopentyl group, a cyclohexyl group, or a menthyl group; and
a compound (2-8) in which Y is a group represented by —N(R⁵)—; R² and R⁵ are independently a tert-butyl group, a tert-pentyl group, a cyclohexyl group, an adamantyl group, a 2,6-dimethylphenyl group, a 2,6-dichlorophenyl group, a 2,6-dibromophenyl group, a 2,4,6-trimethylphenyl group, or a 2,6-diisopropylphenyl group; [symbol]
is a single bond; R⁶ and R⁷ are independently a hydrogen atom, a methyl group, an ethyl group, a propyl group, an isopropyl group, a butyl group, an isobutyl group, a sec-butyl group, a tert-butyl group, a pentyl group, a decyl group, a cyclopropyl group, a 2,2-dimethylcyclopropyl group, a cyclopentyl group, a cyclohexyl group, or a menthyl group.

Examples of the compound (2-8) include 2-carboxy-4,5-dihydro-1,3-di-tert-butylimidazolium, 2-carboxy-4,5-dihydro 1,3-dicyclohexylimidazolium, 2-carboxy-4,5-dihydro-1,3-diadamantylimidazolium, 2-carboxy-4,5-dihydro-1,3-diphenylimidazolium, 2-carboxy-4,5-dihydro-1,3-bis[(2,6-diisopropyl)phenyl]imidazolium, 2-carboxy-4,5-dihydro-1,3-bis[(2,4,6-trimethyl)phenyl]imidazolium, 2-carboxy-4,5-dihydro-4,5-dimethyl-1,3-bis[(2,6-diisopropyl)phenyl]imidazolium, 2-carboxy-4,5-dihydro-4,5-dimethyl-1,3-bis[(2,4,6-trimethyl)phenyl]imidazolium, 2-carboxy-4,5-dihydro-4,5-dichloro-1,3-bis[(2,6-diisopropyl)phenyl]imidazolium, 2-carboxy-4,5-dihydro-4,5-diphenyl-1,3-bis[(2,4,6-trimethyl)phenyl]imidazolium, 2-carboxy-4,5-dihydro-4,5-difluoro-1,3-bis[(2,6-diisopropyl)phenyl]imidazolium, 2-carboxy-4,5-dihydro-4-methyl-1,3-bis[(2,4,6-trimethyl)phenyl]imidazolium, 2-carboxy-4,5-dihydro-1,3-bis[(2,6-dichloro) phenyl]imidazolium, 2-carboxy-4,5-dihydro-1-tert-butyl-3-phenylimidazolium, 2-carboxy-4,5-dihydro-1-cyclohexyl-3-[(2,6-diisopropyl)phenyl]imidazolium, 2-carboxy-4,5-dihydro-1-phenyl-3-[(2,4,6-trimethyl)phenyl]imidazolium, 2-carboxy-4,5-dihydro-1-tert-butyl-3-[(2,6-diisopropyl)phenyl]imidazolium, and 2-carboxy-4,5-dihydro-1-tert-butyl-3-[(2,4,6-trimethyl)phenyl]imidazolium.

Example of the compound (2-9) include

a compound (2-9) in which Y is a group represented by —N(R^{b})—; R² is a linear or branched C₁ to C₁₂ alky group or a C₆ to C₂₀ aryl group; and R⁵ is a linear or branched C₁ to C₁₂ alky group or a C₆ to C₂₀ aryl group;
a compound (2-9) in which Y is a group represented by —N(R⁵)—; R² represents a linear or branched C₁ to C₁₂ alky group or a C₆ to C₂₀ aryl group; R⁵ is a linear or branched C₁ to C₁₂ alky group or a C₆ to C₂₀ aryl group; and R⁷ is a C₁ to C₁₀ alky group or a C₆ to C₁₀ aryl group;
a compound (2-9) in which Y is a group represented by —S— and R² is a linear or branched C₁ to C₁₂ alky group or a C₆ to C₂₀ aryl group; and
a compound (2-9) in which Y is a group represented by —S—; R² is a linear or branched C₁ to C₁₂ alky group or a C₆ to C₂₀ aryl group; and R⁷ is a C₁ to C₁₀ alky group or a C₆ to C₁₀ aryl group.

Examples of the compound (2-9) include 5-carboxy-1,3,4-triphenyl-4H,1,2,4-triazolium.

Examples of the compound (2-2) and the compound (2-3) include commercialized products and those produced according to the method disclosed in, for example, J. Am. Chem. Soc., vol. 127, p. 9079 (2005).

Hereinafter, a method for generating a carbene catalyst by decomposition of the compound (2-2) will be described.

Preferably, the compound (2-2) is decomposed into a carbene catalyst and an alcohol by heating the compound to a prescribed temperature and the alcohol is removed to generate the carbene catalyst.

The generation of the carbene catalyst may be carried out simultaneously with the oxidization reaction in the step A as described below or the carbene catalyst is generated previously and then the carbene catalyst is added to the oxidization reaction in the step A.

In the case of generating the carbene catalyst simultaneously with the oxidization reaction in the step A, a solvent may be used or may not be used. In the case of previously generating the carbene catalyst, the carbene catalyst is preferable to be generated in the presence of a solvent. The solvent preferably used is a solvent that does not react with the generated carbene catalyst and examples thereof include ether solvents such as tetrahydrofuran, methyl tert-butyl ether, cyclopentyl methyl ether, and diisopropyl ether; ester solvents such as ethyl acetate and butyl acetate; aromatic solvents such as toluene and chlorobenzene; nitrile solvents such as acetonitrile and propionitrile; and their solvent mixtures.

The use amount of the solvent is not limited and practically, for example, it is 100 parts by weight or less per 1 part by weight of the compound (2-2).

The mixing order of reaction reagents is not limited in the generation of the carbene catalyst. Examples of a preferable embodiment include methods of mixing the compound (2-2) and the solvent, heating the mixture to be obtained to a prescribed temperature, and removing the alcohol to be generated by distillation.

The generation of the carbene catalyst is carried out in any condition of reduced pressure, normal pressure, and applied pressure and preferably carried out in a condition of normal pressure or applied pressure.

The reaction temperature in the generation of the carbene catalyst is different depending on the type of the compound (2-2), the type of the carbene catalyst to be generated and the like, but is preferably in a range of −20° C. to 100° C. and more preferably in a range of 0° C. to 50° C. In the case where the reaction temperature is lower than −20° C., the generation rate of the carbene catalyst tends to be low and in the case where the reaction temperature is higher than 100° C., the carbene catalyst to be produced tends to be decomposed.

The progression of the reaction in the generation of the carbene catalyst can be confirmed by an analysis means, for example, thin-layer chromatography, nuclear magnetic resonance spectroscopic analysis, infrared absorption spectrometry, etc.

After completion of the reaction in the generation of the carbene catalyst, the reaction solution containing the carbene catalyst can be used as it is for the oxidation reaction of the step A. Further, the reaction mixture be obtained may be, for example, subjected to a concentration treatment if necessary and subsequently subjected to a cooling treatment or the like to take the carbene catalyst out.

Hereinafter, a method for generating a carbene catalyst by decomposition of the compound (2-3) will be described.

Preferably, the compound (2-3) is decomposed into a carbene catalyst and carbon dioxide by heating the compound to a prescribed temperature and carbon dioxide is removed to generate the carbene catalyst.

The generation of the carbene catalyst may be carried out simultaneously with the oxidization reaction in the step A as described below or the carbene catalyst is generated previously and then the carbene catalyst is added to the oxidization reaction in the step A.

In the case of generating the carbene catalyst simultaneously with the oxidization reaction in the step A, a solvent may be used or may not be used. In the case of previously generating the carbene catalyst, the carbene catalyst is preferable to be generated in the presence of a solvent. The solvent preferably used is a solvent that does not react with the generated carbene catalyst and examples thereof include ether solvents such as tetrahydrofuran, methyl tert-butyl ether, cyclopentyl methyl ether, and diisopropyl ether; ester solvents such as ethyl acetate and butyl acetate; aromatic solvents such as toluene and chlorobenzene; nitrile solvents such as acetonitrile and propionitrile; and their solvent mixtures.

The use amount of the solvent is not limited and practically, for example, it is 100 parts by weight or less per 1 part by weight of the compound (2-3).

The mixing order of reaction reagents is not limited in the generation of the carbene catalyst. Examples of a preferable embodiment include methods of mixing the compound (2-3) and the solvent, heating the mixture to be obtained to a prescribed temperature, and removing carbon dioxide.

The generation of the carbene catalyst is carried out in any condition of reduced pressure, normal pressure, and applied pressure and preferably carried out in a condition of normal pressure or applied pressure.

The reaction temperature in the generation of the carbene catalyst is different depending on the type of the compound (2-3), the type of the carbene catalyst to be generated and the like, but is preferably in a range of −20° C. to 100° C. and more preferably in a range of 0° C. to 50° C. In the case where the reaction temperature is lower than −20° C., the generation rate of the carbene catalyst tends to be low and in the case where the reaction temperature is higher than 100° C., the carbene catalyst to be produced tends to be decomposed.

The progression of the reaction in the generation of the carbene catalyst can be confirmed by an analysis means, for example, thin-layer chromatography, nuclear magnetic resonance spectroscopic analysis, infrared absorption spectrometry, etc.

After completion of the reaction in the generation of the carbene catalyst, the reaction solution containing the carbene catalyst can be used as it is for the oxidation reaction of the step A. Further, the reaction mixture to be obtained may be, for example, subjected to a concentration treatment if necessary and subsequently subjected to a cooling treatment or the like to take the carbene catalyst out.

The step A is preferably carried out as described above by causing a reaction of 4-methylthio-2-oxo-1-butanal, an alcohol, and an oxidizing agent in the presence of a carbene catalyst, more preferably by causing a reaction of 4-methylthio-2-oxo-1-butanal, an alcohol, and an oxidizing agent in the presence of at least one selected from the group consisting of a compound obtained by causing a reaction of a compound represented by the formula (2-1) and a base, a compound represented by the formula (2-2), a compound obtained by decomposing the compound represented by the formula (2-2), a compound represented by the formula (2-3), and a compound obtained by decomposing the compound represented by the formula (2-3), and furthermore preferably by causing a reaction of 4-methylthio-2-oxo-1-butanal, an alcohol, and an oxidizing agent in the presence of the compound obtained by causing a reaction of a compound represented by the formula (2-1) and a base.

The use amount of the carbene catalyst is preferably 0.001 mol to 0.5 mol and more preferably 0.01 mol to 0.3 mol per 1 mol of 4-methylthio-2-oxo-1-butanal.

Next, the alcohol to be used in the step A will be described.

In the step A, the type of the alcohol is not limited and a lower alcohol with 1 to 8 carbon atoms is preferably used as the alcohol. Examples thereof include methanol, ethanol, 1-propanol, isopropanol, 1-butanol, isobutanol, 1-pentanol, 1-hexanol, cyclohexanol, and benzyl alcohol. Methanol or ethanol is more preferable.

Examples of the alcohol to be used in the step A include a commercialized product and those produced according to any known methods. Examples of the known method include a method of partially oxidizing alkane or alkyl-substituted benzene; a method for adding water to a double bond; and a fermentation method.

The use amount of the alcohol in the step A is preferably 1 mol or more per 1 mol of 4-methylthio-2-oxo-1-butanal, and the upper limit thereof is not limited, but in terms of economy, it is preferably 100 mol or lower.

The oxidizing agent to be used in the step A is not particularly limited unless it promotes a side reaction preferentially, and examples thereof include oxygen, carbon dioxide, manganese dioxide, azobenzene, quinone, benzoquinone, and anthraquinone. The oxidizing agent is preferably at least one selected from the group consisting of oxygen and carbon dioxide.

The oxygen that can be used as the oxidizing agent in the step A may be oxygen gas, or oxygen gas diluted with an inert gas such as nitrogen, or oxygen contained in atmospheric air. Oxygen contained in atmospheric acid and diluted with an inert gas such as nitrogen may also be used.

The use amount of the oxygen is preferably in a range of 1 mol to 100 mol per 1 mol of 4-methylthio-2-oxo-1-butanal.

The carbon dioxide that can be used as the oxidizing agent in the step A may be gaseous carbon dioxide, solid-phase (dry ice) carbon dioxide and carbon dioxide in a supercritical state. Gaseous carbon dioxide may be diluted with an inert gas such as nitrogen.

The use amount of the carbon dioxide is preferably 1 mol or more per 1 mol of 4-methylthio-2-oxo-1-butanal, and the upper limit thereof is not limited, but in terms of productivity, it may be 100 mol or less.

The step A may be carried out in the presence of a solvent.

Solvents which do not inhibit production of a 4-methylthio-2-oxo-butanoic acid ester in the step A may be used without limitation, and examples thereof include ether solvents such as tetrahydrofuran, methyl tert-butyl ether, cyclopentyl methyl ether, and diisopropyl ether; ester solvents such as ethyl acetate and butyl acetate; aromatic solvents such as toluene and chlorobenzene; nitrile solvents such as acetonitrile and propionitrile; and their solvent mixtures.

The use amount of the solvent is not limited and practically, it is 100 parts by weight or less per 1 part by weight of 4-methylthio-2-oxo-1-butanal.

The mixing order of reaction reagents is not limited in the step A. Examples of a preferable embodiment include, in the case where the compound (2-1) is used, a method of mixing 4-methylthio-2-oxo-1-butanal, the compound (2-1), the oxidizing agent, the alcohol, and if necessary the solvent and adding the base to the mixture to be obtained and, in the case where at least one selected from the group consisting of the compound (2-2) and the compound (2-3), a method of mixing 4-methylthio-2-oxo-1-butanal, the alcohol, at least one compound selected from the group consisting of the compound (2-2) and the compound (2-3), and if necessary the solvent and adding the oxidizing agent to the mixture to be obtained.

The step A is carried out in any condition of reduced pressure, normal pressure, and applied pressure and preferably carried out in a condition of normal pressure or applied pressure.

The reaction temperature in the step A is different depending on the type, use amount and the like of the carbene catalyst and further depending on the type, use amount and the like of the base in the case of using the compound (2-1), but is preferably in a range of −20° C. to 150° C. and more preferably in a range of 0° C. to 100° C. In the case where the reaction temperature is lower than −20° C., the oxidation reaction rate tends to be low and in the case where the reaction temperature is higher than 150° C., selectivity of the reaction tends to be lowered.

The progression of the reaction in the step A can be confirmed by an analysis means, for example, gas chromatography, high performance liquid chromatography, thin-layer chromatography, nuclear magnetic resonance spectroscopic analysis, infrared absorption spectrometry, etc.

After completion of the reaction in the step A, the oxidizing agent used is removed by degassing, filtration or the like, and then the reaction mixture to be obtained may be, for example, subjected to a concentration treatment if necessary and subsequently subjected to a cooling treatment or the like to take a 4-methylthio-2-oxo-butanoic acid ester out.

The 4-methylthio-2-oxo-butanoic acid ester to be taken out may be refined by a refining means such as distillation, column chromatography, crystallization, etc.

Next, the step B of hydrolyzing the 4-methylthio-2-oxo-butanoic acid ester obtained in the step A will be described. Execution of the step B gives 4-methylthio-2-oxo-butanoic acid. 4-Methylthio-2-oxo-butanoic acid may form a salt. The salt means a salt obtained by replacing H⁺ dissociated from a group represented by —COOH contained in 4-methylthio-2-oxo-butanoic acid with any cation.

Examples of the cation include alkali metal ions such as a lithium ion, a sodium ion, and a potassium ion; alkaline earth metal ions such as a calcium ion and a magnesium ion; quaternary ammonium ions such as a tetramethylammonium ion and a tetrabutylammonium ion; and an ammonium ion.

The hydrolysis in the step B may be any of acid hydrolysis and alkaline hydrolysis. In the case of carrying out acid hydrolysis, acids that can be used are, for example, mineral acids such as sulfuric acid, hydrochloric acid, and phosphoric acid; organic sulfonic acids such as methanesulfonic acid and p-toluenesulfonic acid; and an acidic cation exchange resin. In the case of carrying out alkaline hydrolysis, alkalis that can be used are, for example, alkali metal hydroxides such as sodium hydroxide and potassium hydroxide; alkali metal hydrogen carbonates such as sodium hydrogen carbonate and potassium hydrogen carbonate; alkali metal carbonates such as sodium carbonate and potassium carbonate; and ammonia.

The hydrolysis in the step B can be carried out by, for example, mixing the 4-methylthio-2-oxo-butanoic acid ester obtained in the step A with the acid or the alkali in the presence of water. The reaction temperature of the hydrolysis is, for example, in a range of 20° C. to 100° C. and preferably in a range of 40° C. to 80° C. While alcohols produced by hydrolysis are removed by distillation, hydrolysis may be carried out.

After completion of the hydrolysis reaction, the reaction mixture to be obtained may be used as it is for the reductive amination reaction in the step C, or after 4-methylthio-2-oxo-butanoic acid or its salt is taken out, it may be used for the reductive amination reaction in the step C.

After completion of the hydrolysis reaction, the reaction mixture to be obtained may be subjected to, for example, a post-treatment such as filtration, neutralization, extraction, washing with water or the like, and to a isolation treatment such as distillation, crystallization or solid-liquid separation after temperature adjustment if necessary to take 4-methylthio-2-oxo-butanoic acid or its salt out. The isolated 4-methylthio-2-oxo-butanoic acid or its salt may be refined by a refining treatment of recrystallization; extraction refining; distillation; adsorption treatment with activated carbon, silica, alumina, etc.; chromatography such as silica gel column chromatography; or the like.

Next, the step C of reductive amination of 4-methylthio-2-oxo-butanoic acid obtained in the step B will be described. Execution of the step C gives methionine.

The step C is carried out by reductive amination of 4-methylthio-2-oxo-butanoic acid obtained in the step B and it may be carried out by using a reducing agent such as a hydrogenated aluminum alkali metal salt or a hydrogenated boron alkali metal salt or by using hydrogen or formic acid as a reducing agent in the presence of a metal catalyst. The step C is preferably carried out by causing a reaction of 4-methylthio-2-oxo-butanoic acid, ammonia, and a reducing agent in the presence of a transition metal.

The transition metal to be used in the step C (hereinafter, may be referred to as “transition metal catalyst” in some cases) is preferably at least one selected from the group consisting of noble metals, nickel, cobalt, and copper, and also preferably at least one selected from the group consisting of ruthenium, rhodium, palladium, platinum, iridium, nickel, cobalt, and copper.

Examples of the noble metal include ruthenium, rhodium, palladium, platinum, and iridium, and the noble metal is preferably deposited on a support (hereinafter, may be referred to as “supported catalyst” in some cases). The support is preferably at least one selected from the group consisting of, for example, activated carbon, alumina, silica, and zeolite.

Examples of the nickel include reduced nickel (hereinafter, may be referred to as “reduced nickel catalyst” in some cases) and sponge nickel (Raney Nickel (registered trademark) (hereinafter, may be referred to as “sponge nickel catalyst”); examples of the cobalt include reduced cobalt (hereinafter, may be referred to as “reduced cobalt catalyst” in some cases) and sponge cobalt (Raney Cobalt (registered trademark) (hereinafter, may be referred to as “sponge cobalt catalyst”); and examples of the copper include sponge copper (Raney Copper (registered trademark) (hereinafter, may be referred to as “sponge copper catalyst”). Reduced metal catalysts such as the reduced nickel catalyst and the reduced cobalt catalyst are catalysts prepared by reducing a metal oxide or a metal hydroxide, or catalysts prepared by reducing a metal oxide deposited on a support or a metal hydroxide deposited on a support. Sponge metal catalysts such as the sponge nickel catalyst, the sponge cobalt catalyst, and the sponge copper catalyst are catalysts prepared by causing an action of an aqueous sodium hydroxide solution on an alloy of nickel and aluminum, an alloy of cobalt and aluminum, or an alloy of copper and aluminum to melt aluminum.

The transition metal catalyst in the step C is preferably the sponge metal catalyst or the noble metal, and more preferably at least one catalyst selected from the group consisting of the sponge nickel catalyst, the sponge cobalt catalyst, and the sponge copper catalyst, or the noble metal deposited on a support. Among them, the transition metal catalyst is preferably palladium deposited on activated carbon or rhodium deposited on activated carbon.

The transition metal catalyst may be a commercialized product or may be those prepared by any known method.

The use amount of the transition metal catalyst is preferably 2 parts by weight or less of the transition metal atom, more preferably in a range of 0.0001 to 0.2 parts by weight of the transition metal atom, and furthermore preferably in a range of 0.001 to 0.1 parts by weight of the transition metal atom per 1 part by weight of 4-methylthio-2-oxo-butanoic acid.

The ammonia to be used in the step C may be in any form of liquefied ammonia, ammonia gas, and an ammonia solution, and preferably an ammonia solution and more preferably ammonia water or an ammonia methanol solution. In the case where ammonia water is used, the concentration is preferably 10 to 35 wt %. Further, ammonia may form a salt with an inorganic acid such as hydrochloric acid or sulfuric acid, or a carboxylic acid such as formic acid or acetic acid.

The use amount of ammonia is preferably 1 mol or more per 1 mol of 4-methylthio-2-oxo-butanoic acid. The upper limit of the use amount of ammonia is not limited and, for example, 500 mol per 1 mol of 4-methylthio-2-oxo-butanoic acid.

The reducing agent to be used in the step C is preferably at least one selected from the group consisting of hydrogen and formic acid. Hydrogen to be used may be commercialized hydrogen gas, or may be generated from, for example, formic acid or its salt by any known method. In the case where hydrogen gas is used, the partial pressure thereof is preferably 10 MPa or lower, more preferably in a range of 0.01 to 5 MPa, furthermore preferably in a range of 0.02 to 2 MPa, and even more preferably in a range of 0.05 to 0.8 MPa. Formic acid to be used may be, for example, a commercialized product.

The reductive amination in the step C (hereinafter, may be referred to as “reductive amination reaction” in some cases) is preferably carried out in the presence of a solvent. The solvent is preferably inactive to the reductive amination reaction and examples thereof include aliphatic hydrocarbon solvents such as pentane, hexane, isohexane, heptane, isoheptane, octane, isooctane, nonane, isononane, decane, isodecane, undecane, dodecane, cyclopentane, cyclohexane, methylcyclohexane, tert-butylcyclohexane, and petroleum ether; ether solvents such as tetrahydrofuran, methyltetrahydrofuran, diethyl ether, dipropyl ether, diisopropyl ether, dibutyl ether, dipentyl ether, dihexyl ether diheptyl ether, dioctyl ether, tert-butyl methyl ether, cyclopentyl methyl ether, 1,2-dimethoxyethane, and diethylene glycol dimethyl ether; alcohol solvents such as methanol, ethanol, 1-propanol, 2-propanol, 1-butanol, isobutyl alcohol, tert-butyl alcohol, 1-pentanol, 2-pentanol, isopentyl alcohol, 1-hexanol, 2-hexanol, isohexyl alcohol, 1-heptanol, 2-heptanol, 3-heptanol, isoheptyl alcohol, ethylene glycol monomethyl ether, ethylene glycol monoethyl ether, ethylene glycol monopropyl ether, ethylene glycol monoisopropyl ether, ethylene glycol monobutyl ether, ethylene glycol monoisobutyl ether, ethylene glycol mono-tert-butylether, diethylene glycol monomethyl ether, diethylene glycol monoethyl ether, diethylene glycol monopropyl ether, diethylene glycol monoisopropyl ether, diethylene glycol monoethyl ether, diethylene glycol monoisobutyl ether, and diethylene glycol mono-tert-butyl ether; ester solvents such as ethyl acetate, propyl acetate, isopropyl acetate, butyl acetate, isobutyl acetate, tort-butyl acetate, amyl acetate, and isoamyl acetate; aprotic polar solvents such as dimethyl sulfoxide, sulfolane, N,N-dimethylformamide, N,N-dimethylacetamide, N,N-dimethylpropionamide, N-methylpyrrolidone, γ-butyrolactone, dimethyl carbonate, diethyl carbonate, ethylene carbonate, propylene carbonate, 1,3-dimethyl-2-imidazolidinone, and 1,3-dimethyl-3,4,5,6-tetrahydro-2(1H)-pyrridinone; water; and their mixtures. Among them, an alcohol solvent or water is preferable, and methanol or water is more preferable. The use amount of the solvent is preferably in a range of 1 to 200 mL and more preferably in a range of 10 to 150 mL per 1 g of 4-methylthio-2-oxo-butanoic acid.

The mixing order of reaction reagents in the step C is not particularly defined, and examples thereof include a method of mixing 4-methylthio-2-oxo-butanoic acid, ammonia, and the transition metal catalyst and adding hydrogen to the mixture to be obtained; and a method of mixing 4-methylthio-2-oxo-butanoic acid and ammonium formate, adding formic acid if necessary to adjust the pH to an arbitrary value, and subsequently, adding the transition metal catalyst to the mixture to be obtained.

The reaction temperature in the step C is preferably in a range of 0° C. to 100° C. and more preferably in a range of 20° C. to 90° C. The reaction time is, for example, in a range of 1 to 24 hours, although depending on the reaction temperature, the use amount of the reaction reagents and solvent, the hydrogen partial pressure and the like. The progression of the reductive amination reaction can be confirmed by an analysis means such as thin-layer chromatography, gas chromatography, high performance liquid chromatography, etc.

After completion of the reductive amination reaction, the reaction mixture to be obtained may be subjected to, for example, a post-treatment such as filtration, neutralization, extraction, washing with water or the like, and to a isolation treatment such as distillation, crystallization or solid-liquid separation after temperature adjustment if necessary to take methionine out. Specifically, for example, after temperature adjustment to near room temperature or without temperature adjustment, the reaction mixture to be obtained is subjected to filtration to remove the transition metal catalyst and thereafter, the obtained filtrate is neutralized to precipitate methionine, and the precipitated methionine can be recovered by filtration or the like. For example, in the case where the reaction mixture to be obtained is basic, neutralization is carried out by mixing the reaction mixture with an acid such as hydrochloric acid or carbonic acid; and in the case where the reaction mixture to be obtained is acidic, neutralization is carried out by mixing the reaction mixture with a base such as sodium carbonate, sodium hydrogen carbonate, or potassium carbonate. The transition metal catalyst removed by filtration and the methionine recovered by filtration or the like may be washed with the above-mentioned solvent. The recovered methionine may also be dried by vacuum drying or the like. In the case where ammonia is contained in the reaction mixture, ammonia can be removed from the reaction mixture by blowing nitrogen gas to the reaction mixture.

The isolated methionine may be refined by a refining treatment of recrystallization; extraction refining; distillation; adsorption treatment with activated carbon, silica, alumina, etc.; chromatography such as silica gel column chromatography; or the like.

### EXAMPLES

Hereinafter, the present invention will be described by way of examples.

### Production Example of 1,3-bis[(2,4,6-tribromo)phenyl]imidazolinium chloride Serving as Carbene Catalyst Raw Material

### Reference Example 1

A nitrogen-purged 300 mL flask was loaded with 25 g of 2,4,6-tribromoaniline, 200 g of chloroform, and 9.2 g of triethylamine. Oxalyl chloride in an amount of 11.5 g was added dropwise at 0° C. over 30 minutes to the obtained mixture. The obtained mixture was stirred at 0° C. for 2 hours and further stirred at room temperature for 18 hours. Water in an amount of 10.0 g was added to the obtained reaction mixture to precipitate a crystal. Next, the precipitated crystal was recovered by filtration operation and the recovered product was washed with 10 g of water and 20 g of diethyl ether and then further dried to obtain 20.4 g of a white crystal. The obtained white crystal was confirmed to be N,N′-bis(2,4,6-tribromophenyl)ethanediamide by gas chromatography/mass spectrometry (GC-MS). Yield: 76%.

MS (m/z): 713 (M+)

After 10.1 g of the N,N′-bis(2,4,6-tribromophenyl)ethanediamide obtained above and 85 mL of a 1 M BH3-tetrahydrofuran solution were loaded to a 200 mL autoclave made of stainless steel, the mixture was heated and stirred at 75° C. for 16 hours. After cooled to room temperature, the reaction solution was added gradually to a mixed solution of 170 g of methanol and 8.5 g of 35% hydrochloric acid and stirred. Low-boiling point substances were removed by distillation from the obtained reaction solution, 150 g of methanol was further added to the residues, and again the low-boiling point substances were removed by distillation to obtain 9.1 g of a white crystal. Yield: 89%.

The obtained crystal was confirmed to be N,N′-bis(2,4,6-tribromophenyl)-1,2-ethanediamine hydrochloride by GC-MS.

MS (m/z): 685 (M+, free amine)

A nitrogen-purged 200 mL flask was loaded with 9 g of the N,N′-bis(2,4,6-tribromophenyl)-1,2-ethanediamine hydrochloride obtained above and 100 g of triethyl orthoformate and the obtained mixture was refluxed for 1 hour and thereafter cooled to room temperature to precipitate a crystal. Next, the precipitated crystal was recovered by filtration operation and the recovered product was washed with 10 g of tetrahydrofuran and dried to obtain 3.1 g of a white crystal. The obtained crystal was confirmed to be 1,3-bis[(2,4,6-tribromo)phenyl]imidazolinium chloride by ¹H-NMR. Yield: 32%.

¹H-NMR (δ/ppm, DMSO-d6, based on tetramethyl silane): 4.66 (s, 4H), 8.3 (s, 4H), 9.70 (s, 1H)

### Production Example of 4-methylthio-2-oxo-butanoic acid methyl ester

### Step A

### Example 1

A 100 mL pressure-resistant reaction tube made of stainless steel and equipped with a magnetic rotator was loaded with 100 mg of 4-methylthio-2-oxo-1-butanal, 20 mg of 3-(2,6-diisopropyl)phenyl-4,5-dimethylthiazolium chloride, 500 mg of methanol, and 3 g of tetrahydrofuran and while blowing nitrogen gas, the obtained mixture was cooled in a dry ice bath at −70° C. After 2 g of dry ice and 6 mg of sodium methylate were added to the cooled mixture, the pressure-resistant reaction tube was tightly plugged. The obtained mixture was stirred at 60° C. for 4 hours to cause a reaction.

After completion of the reaction, carbon dioxide and carbon monoxide produced as a byproduct, both in a gas state, were removed from the reaction mixture, and then when the obtained reaction mixture was analyzed by an internal standard method in gas chromatography to measure the yield of 4-methylthio-2-oxo-butanoic acid methyl ester, the yield was 50%. In the reaction mixture after completion of the reaction, 10% unreacted 4-methylthio-2-oxo-1-butanal remained.

### Production Example of 4-methylthio-2-oxo-butanoic acid methyl ester

### Step A

### Example 2

A 100 mL pressure-resistant reaction tube made of stainless steel and equipped with a magnetic rotator was loaded with 100 mg of 4-methylthio-2-oxo-1-butanal, 18 mg of 3-(2,6-diisopropyl)phenyl-4,5-dimethylthiazolium chloride, 300 mg of methanol, and 3 g of tetrahydrofuran and while blowing nitrogen gas, the obtained mixture was cooled in a dry ice bath at −70° C. After 2 g of dry ice and 10 mg of a 28% sodium methylate solution in methanol were added to the cooled mixture, the pressure-resistant reaction tube was tightly plugged. The obtained mixture was pressurized at 1 MPa with air and stirred at 60° C. for 3 hours to cause a reaction.

After completion of the reaction, the obtained reaction mixture was cooled to room temperature and then the pressure was allowed to return to normal pressure by pressure discharge, and when the obtained reaction mixture was analyzed by an internal standard method in gas chromatography to measure the yield of 4-methylthio-2-oxo-butanoic acid methyl ester, the yield was 20%. In the reaction mixture after completion of the reaction, 20% unreacted 4-methylthio-2-oxo-1-butanal remained.

### Production Example of 4-methylthio-2-oxo-butanoic acid methyl ester

### Step A

### Example 3

The production was carried out in the same manner as in <Step A-Example 2> except that 36 mg of 1,3-bis[(2,4,6-tribromo)phenyl]imidazolinium chloride was used in place of 18 mg of 3-(2,6-diisopropyl)phenyl-4,5-dimethylthiazolium chloride in <Step A-Example 2>. When the yield of 4-methylthio-2-oxo-butanoic acid methyl ester was measured, the yield was 57%. In the reaction mixture after completion of the reaction, 6% unreacted 4-methylthio-2-oxo-1-butanal remained.

### Production Example of 4-methylthio-2-oxo-butanoic acid methyl ester

### Step A

### Example 4

A 100 mL Schlenk tube equipped with a magnetic rotator was loaded with 100 mg of 4-methylthio-2-oxo-1-butanal, 36 mg of 1,3-bis[(2,4,6-tribromo)phenyl]imidazolinium chloride, 300 mg of methanol, and 3 g of tetrahydrofuran, and after adding 10 mg of a 28% sodium methylate solution in methanol, the obtained mixture was stirred at 60° C. for 3 hours in atmospheric air to cause a reaction.

After completion of the reaction, the obtained reaction mixture was cooled to room temperature, and then when the obtained reaction mixture was analyzed by an internal standard method in gas chromatography to measure the yield of 4-methylthio-2-oxo-butanoic acid methyl ester, the yield was 65%. In the reaction mixture after complexion of the reaction, 4% unreacted 4-methylthio-2-oxo-1-butanal remained.

### Production Example of 4-methylthio-2-oxo-butanoic acid methyl ester

### Step A

### Example 5

A 100 mL Schlenk tube equipped with a magnetic rotator was loaded with 1 g of 4-methylthio-2-oxo-1-butanal, 300 mg of 1,3-bis[(2,4,6-tribromo)phenyl]imidazolinium chloride, 3 g of methanol, and 10 g of tetrahydrofuran, and after adding 70 mg of a 28% sodium methylate solution in methanol, the obtained mixture was stirred at 60° C. for 3 hours in atmospheric air to cause a reaction.

After completion of the reaction, the obtained reaction mixture was cooled to room temperature, and then when the obtained reaction mixture was analyzed by an internal standard method in gas chromatography to measure the yield of 4-methylthio-2-oxo-butanoic acid methyl ester, the yield was 30%. In the reaction mixture after completion of the reaction, 40% unreacted 4-methylthio-2-oxo-1-butanal remained.

### Production Example of 4-methylthio-2-oxo-butanoic acid methyl ester

### Step A

### Example 6

A 100 mL pressure-resistant reaction tube made of stainless steel and equipped with a magnetic rotator was loaded with 100 mg of 4′-methylthio-2-oxo-O-butanal, 20 mg of 2-methoxy-1,3-bis[(2,6-diisopropyl)phenyl]imidazolidine, 500 mg of methanol, and 2 g of tetrahydrofuran in nitrogen atmosphere and the obtained mixture was cooled in a dry ice bath at −70° C. After 2 g of dry ice was added to the cooled mixture, the pressure-resistant reaction tube was tightly plugged. The obtained mixture was stirred at 60° C. for 6 hours to cause a reaction.

After completion of the reaction, carbon dioxide and carbon monoxide produced as a byproduct, both in a gas state, were removed from the reaction mixture, and then when the obtained reaction mixture was analyzed by an internal standard method in gas chromatography to measure the yield of 4-methylthio-2-oxo-butanoic acid methyl ester, the yield was 20%. In the reaction mixture after completion of the reaction, 30% unreacted 4-methylthio-2-oxo-1-butanal remained.

### Production Example of 4-methylthio-2-oxo-butanoic acid methyl ester

### Step A

### Example 7

A 100 mL pressure-resistant reaction tube made of stainless steel and equipped with a magnetic rotator was loaded with 100 mg of 4-methylthio-2-oxo-1-butanal, 10 mg of 2-carboxy-4,5-dihydro-1,3-bis[(2,4,6-trimethyl)phenyl]imidazolium, 500 mg of methanol, and 3 g of tetrahydrofuran in nitrogen atmosphere and the obtained mixture was cooled in a dry ice bath at −70° C. After 2 g of dry ice was added to the cooled mixture, the pressure-resistant reaction tube was tightly plugged. The obtained mixture was stirred at 60° C. for 4 hours to cause a reaction.

After completion of the reaction, carbon dioxide and carbon monoxide produced as a byproduct, both in a gas state, were removed from the reaction mixture, and then when the obtained reaction mixture was analyzed by an internal standard method in gas chromatography to measure the yield of 4-methylthio-2-oxo-butanoic acid methyl ester, the yield was 10%.

### Production Example of 4-methylthio-2-oxo-butanoic acid potassium salt

### Step B

### Example 1

An aqueous 4-methylthio-2-oxo-butanoic acid potassium salt solution is obtained by adding 5 g of an aqueous 10% potassium hydroxide solution to 100 mg of 4-methylthio-2-oxo-butanoic acid methyl ester and removing methanol to be generated while heating and stirring the mixture at 70° C.

Analysis Method in Step C

In the following step C-1 to step C-12, the reaction mixture was analyzed in the following analysis condition by high performance liquid chromatography (manufactured by SHIMADZU CORPORATION) and conversion ratio and selectivity were calculated according to the following expressions. In the step C-12, methionine prepared separately was used as an internal standard substance and the content of methionine was determined by an internal standard method in high performance liquid chromatography.

### <Analysis Condition>

LC column: Lichrosorb-RP-8

Column temperature: 40° C.

Moving phase: acetonitrile/water=5/95
Additive Sodium 1-pentanesulfonate
Additive concentration 2.5 mmol/L
pH of moving phase 3 (adjusted by adding 40% phosphoric acid)

Flow speed: 1.5 mL/minute

Detection wavelength: 210 nm

Measurement time: 60 minutes

### <Calculation of Conversion Ratio>

Conversion ratio (%)=100(%)−(peak surface area (%) of 4-methylthio-2-oxo-butanoic acid)

### <Calculation of Selectivity>

Selectivity (%)=(peak surface area of methionine)/(peak surface area of entire products)×100

### Production Example of Methionine

### Step C

### Example 1

An autoclave with 60 ml inner capacity was loaded with 50 mg of 4-methylthio-2-oxo-butanoic acid sodium salt, 12.6 mL of a 7 mol/L ammonia methanol solution, and 32 mg of 5 wt % Pd/C (produced by Wako Pure Chemical Industries, Ltd.) and the obtained mixture was stirred. After the autoclave was filled with hydrogen by pressure to increase the pressure to 0.5 MPaG (gauge pressure), that is, the hydrogen gas partial pressure to 0.5 MPa, the mixture was heated to 57° C. and stirred for 6 hours. When a portion of the obtained reaction mixture was analyzed by high performance liquid chromatography, the conversion ratio of 4-methylthio-2-oxo-butanoic acid sodium salt was 99% or higher and the selectivity of methionine was 90%.

### Production Example of Methionine

### Step C

### Example 2

An autoclave with 67 mL inner capacity was loaded with 50 mg of 4-methylthio-2-oxo-butanoic acid sodium salt, 12.6 mL of a 7 mol/L ammonia methanol solution, and 32 mg of 5 wt % Pd/C (produced by Wako Pure Chemical Industries, Ltd.) and the obtained mixture was stirred. The mixture was stirred at 50° C. for 6 hours in hydrogen atmosphere (normal pressure). When a portion of the obtained reaction mixture was analyzed by high performance liquid chromatography, the conversion ratio of 4-methylthio-2-oxo-butanoic acid sodium salt was 99% or higher and the selectivity of methionine was 83%.

### Production Example of Methionine

### Step C

### Example 3

An autoclave with 60 mL inner capacity was loaded with 50 mg of 4-methylthio-2-oxo-butanoic acid sodium salt, 5.4 g of 28 wt % ammonia water, and 32 mg of 5 wt % Pd/C (produced by Wako Pure Chemical Industries, Ltd.) and the obtained mixture was stirred. After the autoclave was filled with hydrogen by pressure to increase the pressure to 1.0 MPaG (gauge pressure), that is, the hydrogen gas partial pressure to 1.0 MPa, the mixture was heated to 50° C. and stirred at 50° C. for 6 hours. When a portion of the obtained reaction mixture was analyzed by high performance liquid chromatography, the conversion ratio of 4-methylthio-2-oxo-butanoic acid sodium salt was 99% or higher and the selectivity of methionine was 34%.

### Production Example of Methionine

### Step C

### Example 4

An autoclave with 60 mL inner capacity was loaded with 44 mg of 4-methylthio-2-oxo-butanoic acid, 5.4 g of 28 wt % ammonia water, and 32 mg of 5 wt % Pd/C (produced by Wake Pure Chemical Industries, Ltd.) and the obtained mixture was stirred. After the autoclave was filled with hydrogen by pressure to increase the pressure to 0.5 MPaG (gauge pressure), that is, the hydrogen gas partial pressure to 0.5 MPa, the mixture was heated to 50° C. and stirred for 6 hours. When a portion of the obtained reaction mixture was analyzed by high performance liquid chromatography, the conversion ratio of 4-methylthio-2-oxo-butanoic acid was 9.9% or higher and the selectivity of methionine was 70%.

### Production Example of Methionine

### Step C

### Example 5

A 10 mL flask was loaded with 50 mg of 4-methylthio-2-oxo-butanoic acid sodium salt, 370 mg of ammonium formate, and 5.0 g of water and formic acid was added to the obtained mixture to adjust the pH to 5.0. After adding 60.5 mg of 5 wt % Rh/C (produced by Wako Pure Chemical Industries, Ltd.), the mixture was heated to 00° C. and stirred at the same temperature for 15 hours. When a portion of the obtained reaction mixture was analyzed by high performance liquid chromatography, the conversion ratio of 4-methylthio-2-oxo-butanoic acid sodium salt was 84% and the selectivity of methionine was 42%.

### Production Example of Methionine

### Step C

### Example 6

An autoclave with 50 mL inner capacity was loaded with 51 mg of 4-methylthio-2-oxo-butanoic acid sodium salt and 5.4 g of 28 wt % ammonia water, and the obtained mixture was stirred, and then 51 mg (wet weight) of Raney (registered trademark) nickel (produced by Degussa) was added to the mixture. After the autoclave was filled with hydrogen by pressure to increase the pressure to 0.5 MPaG (gauge pressure), that is, the hydrogen gas partial pressure to 0.5 MPa, the mixture was heated to 50° C. and stirred for 6 hours. When a portion of the obtained reaction mixture was analyzed by high performance liquid chromatography, the conversion ratio of 4-methylthio-2-oxo-butanoic acid sodium salt was 94% and the selectivity of methionine was 10%.

### Production Example of Methionine

### Step C

### Example 7

An autoclave with 50 mL inner capacity was loaded with 51 mg of 4-methylthio-2-oxo-butanoic acid sodium salt and 5.4 g of 28 wt % ammonia water, and the obtained mixture was stirred, and then 51 mg (wet weight) of Raney (registered trademark) cobalt (produced by Aldrich) was added to the mixture. After the autoclave was filled with hydrogen by pressure to increase the pressure to 0.5 MPaG (gauge pressure), that is, the hydrogen gas partial pressure to 0.5 MPa, the mixture was heated to 50° C. and stirred for 6 hours. When a portion of the obtained reaction mixture was analyzed by high performance liquid chromatography, the conversion ratio of 4-methylthio-2-oxo-butanoic acid sodium salt was 96% and the selectivity of methionine was 27%.

### Production Example of Methionine

### Step C

### Example 8

An autoclave with 50 mL inner capacity was loaded with 51 mg of 4-methylthio-2-oxo-butanoic acid sodium salt and 5.4 g of 28 wt % ammonia water, and the obtained mixture was stirred, and then 51 mg (wet weight) of Raney (registered trademark) copper (produced by Strem Chemicals Inc.) was added to the mixture. After the autoclave was filled with hydrogen by pressure to increase the pressure to 0.5 MPaG (gauge pressure), that is, the hydrogen gas partial pressure to 0.5 MPa, the mixture was heated to 50° C. and stirred for 6 hours. When a portion of the obtained reaction mixture was analyzed by high performance liquid chromatography, the conversion ratio of 4-methylthio-2-oxo-butanoic acid sodium salt was 93% and the selectivity of methionine was 9.5%.

### Production Example of Methionine

### Step C

### Example 9

An autoclave with 60 mL inner capacity was loaded with 50 mg of 4-methylthio-2-oxo-butanoic acid sodium salt and 12.6 mL of a 7 mol/L ammonia methanol solution, and the obtained mixture was stirred, and then 51 mg (wet weight) of Raney (registered trademark) nickel (produced by Degussa) was added to the mixture. After the autoclave was filled with hydrogen by pressure to increase the pressure 0.5 MPaG (gauge pressure), that is, the hydrogen gas partial pressure to 0.5 MPa, the mixture was heated to 50° C. and stirred for 6 hours. When a portion of the obtained reaction mixture was analyzed by high performance liquid chromatography, the conversion ratio of 4-methylthio-2-oxo-butanoic acid sodium salt was 95% and the electivity of methionine was 50%.

### Production Example of Methionine

### Step C

### Example 10

An autoclave with 60 mL inner capacity was loaded with 50 mg of 4-methylthio-2-oxo-butanoic acid sodium salt and 12.6 mL of a 7 mol/L ammonia methanol solution, and the obtained mixture was stirred, and then 51 mg (wet weight) of Raney (registered trademark) cobalt (produced by Aldrich) was added to the mixture. After the autoclave was filled with hydrogen by pressure to increase the pressure to 0.5 MPaG (gauge pressure), that is, the hydrogen gas partial pressure to 0.5 MPa, the mixture was heated to 50° C. and stirred for 6 hours. When a portion of the obtained reaction mixture was analyzed by high performance liquid chromatography, the conversion ratio of 4-methylthio-2-oxo-butanoic acid sodium salt was 91% and the selectivity of methionine was 52%.

### Production Example of Methionine

### Step C

### Example 11

An autoclave with 60 mL inner capacity was loaded with 50 mg of 4-methylthio-2-oxo-butanoic acid sodium salt and 12.6 mL of a 7 mol/L ammonia methanol solution, and the obtained mixture was stirred, and then 51 mg (wet weight) of Raney (registered trademark) copper (produced by Strem Chemicals Inc.) was added to the mixture. After the autoclave was filled with hydrogen by pressure to increase the pressure to 0.5 MPaG (gauge pressure), that is, the hydrogen gas partial pressure to 0.5 MPa, the mixture was heated to 50° C. and stirred for 6 hours. When a portion of the obtained reaction mixture was analyzed by high performance liquid chromatography, the conversion ratio of 4-methylthio-2-oxo-butanoic acid sodium salt was 82% and the selectivity of methionine was 13%.

### Production Example of Methionine

### Step C

### Example 12

An autoclave with 60 mL inner capacity was loaded with an aqueous 4-methylthio-2-oxo-butanoic acid potassium salt solution (2.116 g, content 14.5%), 1.58 g of 28 wt % ammonia water, and 95 mg of 5 wt % Pd/C (produced by Wako Pure Chemical Industries, Ltd.) and the obtained mixture was stirred. After the autoclave was filled with hydrogen by pressure to increase the pressure to 0.5 MPaG (gauge pressure), that is, the hydrogen gas partial pressure to 0.5 MPa, the mixture was heated to 40° C. and stirred for 13 hours. After the obtained reaction mixture was cooled to room temperature and the pressure in the autoclave was discharged to allow the pressure to return to normal pressure, and then the reaction mixture was filtered and the solid matter removed by filtration was washed with water. The methionine content in 7.921 g of a solution obtained by mixing the filtrate and a washing solution was analyzed by an internal standard method in high performance chromatography, and when the production ratio of methionine was measured, it was 72.9%.

Next, carbonic acid gas (CO₂ gas) was blown for 30 minutes to the obtained solution to precipitate a solid.

The precipitated solid was recovered by filtration and the recovered filtrate was washed with 0.5 g of water and dried under reduced pressure to obtain 0.121 g of methionine (content 96%, yield 68%).

The present invention is industrially applicable as a methionine production method.

1. A method for producing methionine comprising
a step A of oxidizing 4-methylthio-2-oxo-1-butanal in the presence of an alcohol;
a step B of hydrolyzing a 4-methylthio-2-oxo-butanoic acid ester obtained in the step A; and
a step C of subjecting 4-methylthio-2-oxo-butanoic acid obtained in the step B to reductive amination.

2. The method according to claim 1, wherein the step A is carried out by causing a reaction of 4-methylthio-2-oxo-1-butanal, an alcohol, and an oxidizing agent in the presence of a carbene catalyst.

3. The method according to claim 2, wherein the carbene catalyst in the step A is at least one selected from the group consisting of:
a compound obtained by a reaction of a compound represented by a formula (2-1) 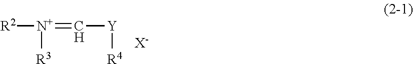
wherein R² represents an optionally substituted alkyl group or an optionally substituted aryl group; R³ and R⁴ each independently represent an optionally substituted alkyl group or an optionally substituted aryl group or R³ and R⁴ may be bonded together to form an optionally substituted divalent hydrocarbon group or an optionally substituted group represented by —CH═N—; Y represents a group represented by —S— or a group represented by —N(R⁵)—; R⁵ represents an optionally substituted alkyl group or an optionally substituted aryl group or R⁵ may be bonded together with R⁴ to form an optionally substituted divalent hydrocarbon group; and X⁻ represents an anion
and a base;
a compound represented by a formula (2-2) 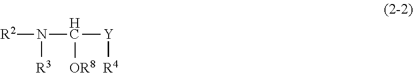
wherein R², R³, R⁴, and Y are the same as described above, respectively; and R⁸ represents an alkyl group;
a compound obtained by decomposing the compound represented by the formula (2-2);
a compound represented by a formula (2-3) 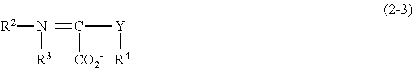
wherein, R², R³, R⁴, and Y are the same as described above, respectively); and
a compound obtained by decomposing the compound represented by the formula (2-3).

4. The method according to claim 2 or 3, wherein the oxidizing agent in the step A is at least one selected from the group consisting of oxygen and carbon dioxide.

5. The method according to claim 1, wherein the alcohol is methanol or ethanol.

6. The method according to claim 1, wherein the step C is carried out in the presence of a solvent.

7. The method according to claim 6, wherein the solvent in the step C is at least one selected from the group consisting of methanol and water.

8. The method according to claim 1, wherein the step C is carried out by causing a reaction of 4-methylthio-2-oxo-butanoic acid, ammonia, and a reducing agent in the presence of a transition metal.

9. The method according to claim 8, wherein the transition metal in the step C is at least one selected from the group consisting of ruthenium, rhodium, palladium, platinum, iridium, nickel, cobalt, and copper.
